SEQUENCE LISTING (9 sequences)

SEQ ID NO: 1 - DNA, 24 bp, Artificial Sequence
  Synthetic
tcgcctactcctcagttagccaca

SEQ ID NO: 2 - DNA, 23 bp, Artificial Sequence
  Synthetic
tgatgatgtgaggccatgtgcga

SEQ ID NO: 3 - DNA, 25 bp, Artificial Sequence
  Synthetic
taatgaaggacttggcagatgaact

SEQ ID NO: 4 - DNA, 20 bp, Artificial Sequence
  Synthetic
acggctttctccctcttgct

SEQ ID NO: 5 - DNA, 116 bp, Artificial Sequence
  Synthetic
tcgcctactcctcagttagccacatagcacttgttattgcatcaatcataatccaaactccatgaagcttcataggagcaacaatactaataatcgcacatggcctcacatcatca

SEQ ID NO: 6 - DNA, 23 bp, Artificial Sequence
  Synthetic
tgatgatgtgaggccatgtgcga

SEQ ID NO: 7 - DNA, 25 bp, Artificial Sequence
  Synthetic
taatgaaggacttggcagatgaact

SEQ ID NO: 8 - DNA, 20 bp, Artificial Sequence
  Synthetic
acggctttctccctcttgct

SEQ ID NO: 9 - DNA, 116 bp, Artificial Sequence; identical to SEQ ID NO: 5
  Synthetic

(19) United States
(12) Patent Application Publication
(10) Pub. No.: US 2022/0354887 A1
(43) Pub. Date: Nov. 10, 2022

(54) METHODS OF PREPARING FUNCTIONAL MITOCHONDRIA AND USES THEREOF

(71) Applicant: UNIVERSITY OF CONNECTICUT, Farmington, CT (US)

(72) Inventors: George Y. WU, Avon, CT (US); Catherine H. WU, Avon, CT (US); Nagham FARAH, New Britain, CT (US); Xiaocong LIU, Farmington, CT (US)

(21) Appl. No.: 17/736,677

(22) Filed: May 4, 2022

Related U.S. Application Data
(60) Provisional application No. 63/185,662, filed on May 7, 2021.

(51) Int. Cl. A61K 35/12; C12N 1/06; C12N 5/071

(52) U.S. Cl. CPC A61K 35/12; C12N 1/06; C12N 5/0602; C12N 2509/00

(57) ABSTRACT

Disclosed herein are methods for introducing functional mitochondria into liver cells in living animals (e.g., mammals). The disclosed compositions and methods can be used to treat clinical conditions characterized by genetic or acquired mitochondrial defects and the resulting dysfunctions and diseases therefrom.

### CROSS-REFERENCE

This application claims priority to U.S. Provisional Patent Application Ser. No. 63/185,662 filed May 7, 2021, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Apr. 26, 2022 having the file name “22-0562-US-SeqList_ST25.txt” and is 2 kb in size.

### BACKGROUND

As the major energy source of mammalian cells, mitochondria have been the subject of numerous studies. However, the isolation and purification of healthy mitochondria especially from fresh tissue remains challenging. The most popular methods and kits involve various centrifugation steps that require substantial time and equipment but do not consistently provide pure preparations of functional mitochondria for use in animals. Therefore, there remains a need in the art to develop methods to improve the purity and yield of functional mitochondria from fresh tissue and deliver them intact to live animals.

### SUMMARY

Disclosed herein are methods for introducing functional mitochondria into liver cells in living animals (e.g., mammals). The disclosed compositions and methods can be used to treat clinical conditions characterized by genetic or acquired mitochondrial defects and the resulting dysfunctions and diseases therefrom, such as, for example, liver damage and liver failure.

In one aspect, disclosed herein is a mitochondrial-protein complex comprising isolated mitochondria; and asialoorosomucoid-poly-L-lysine (AsOR-PL) conjugate or transferrin-poly-L-lysine (Tf-PL) conjugate. In some embodiments, the mitochondrial-protein complex has a mean particle diameter of about 500 nm. In some embodiments, the isolated mitochondria are extracted from mammalian cells. In certain embodiments, the mammalian cells are liver cells. In some embodiments, the mammalian cells are selected from human, porcine, canine, rodent, and feline cells. In particular embodiments, the mammalian cells are rodent cells, such as rat cells or mouse cells.

In some embodiments, the mitochondrial-protein complex is prepared as a pharmaceutical composition. In some embodiments, the pharmaceutical composition comprises one or more pharmaceutically acceptable excipients.

In one aspect, disclosed herein are methods of transplanting mitochondria into a subject, wherein the method comprises administering mitochondrial-protein complex comprising isolated mitochondria and asialoorosomucoid-poly-L-lysine (AsOR-PL) conjugate or transferrin-poly-L-lysine (Tf-PL) conjugate, or a pharmaceutical composition thereof, to a subject in need thereof. In some embodiments, the subject is a mammal. In some embodiments, the method comprises administering the mitochondrial-protein complex by infusion.

Also disclosed herein are methods of treating a mitochondrial dysfunctional disorder, wherein the method comprises administering the disclosed mitochondrial-protein complex, or a pharmaceutically acceptable composition thereof, to a subject in need thereof.

In some embodiments, the mitochondrial dysfunctional disorder is a neuropsychiatric disorder. In some embodiments the neuropsychiatric dysfunction disorder is selected from bipolar disorder (BD), schizophrenia, depression, anxiety disorders, attention deficit disorders, addictive disorders, personality disorders, autism, and Asperger's disease.

In some embodiments, the mitochondrial dysfunctional disorder is a neurodegenerative disorder. In some embodiments, the neurodegenerative disorder is selected from Friedrich's ataxia, human deafness dystonia, amyotrophic lateral sclerosis, Parkinson's disease, Alzheimer's disease, Huntington's disease, and bipolar disorder (BD).

In some embodiments, the mitochondrial dysfunctional disorder is diabetes. In some embodiments, the mitochondrial dysfunctional disorder is juvenile diabetes. In other embodiments, the mitochondrial dysfunctional disorder is type II diabetes.

In some embodiments, the mitochondrial dysfunctional disorder is mitochondrial toxicity associated with therapeutic agents. In some embodiments, the therapeutic agent is selected from a reverse transcriptase inhibitor, a protease inhibitor, and an inhibitor of dihydroorotate dehydrogenase (DHOD). In certain embodiments, the reverse transcriptase inhibitor is selected from azidothymidine (AZT), stavudine (D4T), zalcitabine (ddC), didanosine (DDI), fluoroiodoarauracil (FIAU), lamivudine (3TC), abacavir, and combinations thereof. In other embodiments, the protease inhibitor is selected from ritonavir, indinavir, saquinavir, nelfinavir, and combinations thereof. In yet other embodiments, the inhibitor of dihydroorotate dehydrogenase (DHOD) is leflunomide, brequinar, or a combination thereof.

In some embodiments, the mitochondrial dysfunctional disorder is a migraine.

In some embodiments, the mitochondrial dysfunctional disorder is an ocular disorder associated with mitochondrial dysfunction. In some embodiments, the ocular disorder associated with mitochondrial dysfunctional is selected from glaucoma, diabetic retinopathy, and age-related macular degeneration.

In some embodiments, the mitochondrial dysfunctional disorder is an ischemia-related condition. In some embodiments, the ischemic related condition is selected from angina, stroke, peripheral artery disease, and mesenteric ischemia.

In another aspect, disclosed herein is a method of increasing muscle performance, wherein the method comprises administering a mitochondrial-protein complex comprising isolated mitochondria; and transferrin-poly-L-lysine (Tf-PL) conjugate, to a subject in need thereof. In some embodiments, the method comprises improving the subject's physical endurance. In other embodiments, the method comprises inhibiting the subject's physical fatigue. In yet other embodiments, the method comprises enhancing the subject's blood oxygen levels. In still other embodiments, the method comprises increasing the subject's muscle ATP levels. In other embodiments, the method comprises reducing the level of lactic acid in the subject's blood.

In another aspect, disclosed herein is a composition mitochondrial-protein complex and an endosomal-escape protein, wherein the mitochondrial-protein complex comprises isolated mitochondria and asialoorosomucoid-poly-L-lysine (AsOR-PL) conjugate or transferrin-poly-L-lysine (Tf-PL) conjugate. In some embodiments, the composition further comprises listeriolysin or another targetable endosomolytic agent.

In some embodiments, the endosomolytic protein is prepared by conjugating a pore-forming peptide to a carrier protein. In some embodiments, the pore-forming peptide is a bacterial, viral, or synthetic endosomolytic agent. In some embodiments, the carrier protein is AsOR. In some embodiments, the endosomal endosomolytic protein is prepared by linking a bacterial protein, listeriolysin O (LLO) to AsOR. In some embodiments, the molar ratio of listeriolysin O (LLO) to AsOR is 2:1.

In another aspect, disclosed herein is a method of delivering mitochondria to a subject's liver, wherein the method comprises administering a mitochondrial-protein complex and AsOR-listeriolysin (LLO) to a subject in need thereof, wherein the mitochondrial-protein complex comprises comprising isolated mitochondria and asialoorosomucoid-poly-l-lysine (AsOR-PL) conjugate. In some embodiments, the subject is another mammal.

In another aspect, disclosed herein are methods of treating a liver disease, wherein the method comprises administering a mitochondrial-protein complex and AsOR-listeriolysin (LLO) to a subject in need thereof, wherein the mitochondrial-protein complex comprises isolated mitochondria and asialoorosomucoid-poly-l-lysine (AsOR-PL) conjugate. In some embodiments, the condition is a liver disease of compromised mitochondrial function. In some embodiments, the liver disease is selected from liver steatosis, nonalcoholic steatohepatitis (NASH), fibrosis, cirrhosis, alcoholic liver disease (ALD), liver cell lysis, cholestasis, steatosis, steatohepatitis, cirrhosis, liver cancer, viral hepatitis, and hepatocellular carcinoma (HCC). In some embodiments, the method comprises administering the mitochondrial-protein complex and AsOR-listeriolysin (LLO) to the subject by intravenous infusion.

In another aspect, disclosed herein is a method of preparing a mitochondrial-protein complex comprising mixing isolated mitochondria; and asialoorosomucoid-poly-l-lysine (AsOR-PL) conjugate or transferrin-poly-1-lysine (Tf-PL) conjugate. In some embodiments, the mitochondrial-protein complex has a mean particle diameter of about 500 nm. In some embodiments, the isolated mitochondria are prepared by a method comprising centrifugation of cells at lower speed; centrifugation of cells at higher speed; and filtration of cells through a membrane. In some embodiments, centrifugation at lower speed comprises centrifuging the mitochondria at 1,000×g. In some embodiments, centrifugation at higher speed comprises centrifuging the mitochondria at 7,000×g. In some embodiments, the membrane is a 1.2μ membrane filter. In other embodiments, the membrane is a 0.8μ membrane filter. In some embodiments, the method comprises a two-step filtration process. For example, in some embodiments, the mitochondria first pass through a 1.2μ membrane filter, then, in a second filtration step, the mitochondria pass through a 0.8μ membrane filter. In some embodiments of preparing a mitochondrial-protein complex, the method further comprises the step of rupturing mammalian endosomal membranes. In preferred embodiments, such mammalian cells are mammalian liver cells, such as human, porcine, canine, rodent, and feline liver cells. More preferably, the cells are rat or mouse liver cells.

### BRIEF DESCRIPTION OF THE FIGURES

FIG. 1 shows an image of an agarose gel electrophoresis showing migration of starting materials and AsOR-PL conjugates composed of varying ratios of polylysine (PL) to asialoorosomucoid (AsOR).

FIG. 2 is a bar graph showing the hemolytic activity of AsOR-LLO under physiological and acidic conditions.

FIG. 3 is a graph showing oxygen consumption rate (pmol/min) of the mitochondrial DNA after the standard high-speed spin preparation as a function of time (min). Mitochondrial DNA present was determined by quantitative real time polymerase chain reaction (qPCR) and expressed as means±standard deviation (SD).

FIGS. 4A and 4B show the quantitative effects on of mitochondrial preparation purity before filtration (FIG. 4A) and after filtration (FIG. 4B) as determined by transmission electron microscopy (EM) (per microscopic field at 5000×).

FIG. 4C is a bar graph comparing the mean number of mitochondria identified per field only centrifugation versus centrifugation and filtration.

FIG. 5 is a graph showing the standard curve for mouse mitochondria primers for the calculation of mitochondrial DNA copy number.

FIG. 6 is graph showing the standard curve obtained from qPCR of amplified fragment of standard mouse mitochondrial DNA using mouse specific primers.

FIGS. 7A and 7B are graphs showing the specificity of mouse primers in qPCR of equal copies of mouse and rat mitochondrial DNA.

FIGS. 8A and 8B are transmission electron microscopic images of purified mitochondria. FIG. 8A shows mitochondria after purification but before complexation. FIG. 8B shows purified mitochondria after complexation with AsOR-PL. Magnification 19 200×.

FIG. 9A is a bar graph showing the effects of AsOR-PL in mitochondrial complexes on the integrity and function of mitochondria. Freshly purified mitochondria alone or as AsOR-PL complexes were incubated with IB or DMEM for 1 h and assayed for integrity of mitochondrial outer membrane as determined by cytochrome c oxidase assay and expressed as means relative to mitochondria alone in IB±standard deviation. N.S., no significance. **P<0.01.

FIG. 9B is a graph showing oxygen consumption rate of freshly purified mitochondria alone or as AsOR-PL complexes as a function of time. Freshly purified mitochondria alone or as AsOR-PL complexes were incubated with IB and oxygen consumption was measured and expressed in units of pmol/min normalized to the amount of mitochondrial DNA present as determined by qPCR and expressed as means±SD.

FIG. 10 is a bar graph showing the organ distribution of donor mitochondria. Mouse mitochondria alone, complexed, or complexed mixed with AsOR-LLO were injected intravenously into rats. After 1 h, organs were removed, and DNA was extracted. Using specific primers, qPCR was performed, the percentages of injected mouse mitochondria DNA present in the liver, lung, and spleen were calculated according to sample weight and organ weight. *P<0.05, **P<0.01, compared with the mitochondria alone group.

FIG. 11 is a bar graph showing donor mitochondrial DNA per rat cell. Mitochondria DNA fold-change related to LDHA in rat liver was measured by qPCR. Mouse mitochondria alone, complexed, or complexed mixed with AsOR-LLO were injected intravenously into rats. After 1 h, organs were removed, and DNA was extracted and analyzed by qPCR normalized to LDHA (rat cellular DNA). **P<0.01 compared with the control group.

FIG. 12 is a bar graph showing quantitation of donor mitochondrial DNA in recipient (rat) liver 2 h and 24 h after injection. Mitochondria DNA fold-change related to LDHA in rat liver was measured by qPCR. Mouse mitochondria alone, complexed, or complexed mixed with AsOR-LLO were injected intravenously into rats. Livers were removed, and DNA was extracted and analyzed by qPCR normalized to LDHA (rat cellular DNA).

FIG. 13 is an image of an agarose gel showing purified Tf-PL conjugate.

FIG. 14 is an image of a 7.5% PAGE gel of unpurified Tf-LLO reaction mixture compared to individual reaction mixture components.

FIG. 15 is a bar graph showing mouse hepatoma tissue culture (HTC) mitochondrial DNA fold-changes after incubation of HTC mitochondria complexed to Tf-PL carrier for 2 hrs in the presence of human Huh 7 cells at 37° C. and 4° C.

### DETAILED DESCRIPTION

Mitochondria are the powerhouses of almost all eukaryotic cells, generating about 90% of the chemical energy needed for cell viability. They also possess copies of mitochondrial specific DNA that allow them to replicate as needed by the host cells. However, mitochondria are fragile organelles and have specific intracellular requirements to function optimally. The host cell provides essential nutrients as well as proteins and transcripts encoded by the genomic DNA. Hence, the isolation and purification of healthy viable mitochondria have been challenging tasks. The types of buffers used, and the time spent in the presence of those buffers have been shown to influence the quality of the mitochondrial preparations. For this reason, several kits have been produced containing proprietary buffers. In addition to such buffer systems, most methods to purify mitochondria rely on density gradient ultracentrifugation. However, these methods require specialized equipment, and involve long spin times during which mitochondrial integrity can be compromised. Therefore, even with the use of commercial kits, the quality, the viability, and the yield of mitochondria are inconsistent.

Provided herein is a description of a mitochondrial isolation method using simple high-speed spin steps and a sequential filtration to provide isolated uniform highly purified and functional mitochondria from fresh tissue. In particular, the two-step method provided herein comprises sequential centrifugation followed by membrane filtration results in preparations of highly purified, uniform, and functional mitochondria.

Also disclosed herein are methods for introducing functional mitochondria into liver cells in living animals (e.g., mammals). The disclosed compositions and methods can be used to treat clinical conditions characterized by genetic or acquired mitochondrial defects and the resulting dysfunctions and diseases therefrom.

### Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the invention.

The terms “a,” “an,” and “the” and similar referents used in the context of describing the invention (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to carboxy orientation.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

As used herein, the term “about” refers to a range of values of plus or minus 10% of a specified value. For example, the phrase “about 200” includes plus or minus 10% of 200, or from 180 to 220, unless clearly contradicted by context.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

The terms “isolated,” “purified,” or “biologically pure” refer to material that is substantially or essentially free of components that normally accompany it as found in its native state.

The terms “modulate,” “modulation,” or “modulating” are art-recognized and refer to up-regulation (i.e., activation, stimulation, increase), or down-regulation (i.e., inhibition, suppression, reduction, or decrease) of a response, or the two in combination or apart.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “substantially decreased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has a decrease of at least 10%, preferably about 20%, more preferable about 40%, even more preferable about 50% and still more preferably a decrease of more than 75% when compared to the level, amount, or concentration of the same chemical compound, nucleic acid, polypeptide or physical parameter in a control sample.

As used herein, the term “substantially increased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has an increase of at least 30%, preferably about 50%, more preferable about 75%, and still more preferably an increase of more than 100% when compared to the level, amount, or concentration of the same chemical compound, nucleic acid, polypeptide, or physical parameter in a control sample.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

1. A mitochondrial-protein complex comprising:
isolated mitochondria; and
asialoorosomucoid-poly-L-lysine (AsOR-PL) conjugate or transferrin-poly-L-lysine (Tf-PL) conjugate.

2. The mitochondrial-protein complex of claim 1, having a mean particle diameter of about 500 nm.

3. The mitochondrial-protein complex of claim 1 or 2, wherein the isolated mitochondria are extracted from mammalian cells.

4. The mitochondrial-protein complex of claim 3, wherein the mammalian cells are liver cells.

5. The mitochondrial-protein complex of claim 3 or 4, wherein the mammalian cells are selected from human cells, porcine cells, canine cells, rodent cells, and feline cells.

6. The mitochondrial-protein complex of any one of claims 3-5, wherein the mammalian cells are rodent cells.

7. The mitochondrial-protein complex of any one of claims 3-6, wherein the mammalian cells are rat cells or mouse cells.

8. A pharmaceutical composition comprising the mitochondrial-protein complex of any one of claims 1-7.

9. The pharmaceutical composition of claim 8, wherein the pharmaceutical composition comprises one or more pharmaceutically acceptable excipients.

10. A method of transplanting mitochondria into subject, wherein the method comprises administering the mitochondrial-protein complex of any one of claims 1-7, or the pharmaceutical composition of claim 8 or 9, to a subject in need thereof.

11. The method of claim 10, wherein the subject is a mammal.

12. The method of claim 10 or 11, wherein the method comprises administering the mitochondrial-protein complex by intravenous infusion.

13. A method of treating a mitochondrial dysfunctional disorder, wherein the method comprises administering a mitochondrial-protein complex of any one of claims 1-7, or the pharmaceutical composition of claim 8 or 9, to a subject in need thereof.

14. The method of treating a mitochondrial dysfunctional disorder of claim 13, wherein the mitochondrial dysfunction disorder is a neuropsychiatric disorder.

15. The method of treating a mitochondrial dysfunctional disorder of claim 14, wherein the neuropsychiatric dysfunction disorder is selected from bipolar disorder (BD), schizophrenia, depression, anxiety disorders, attention deficit disorders, addictive disorders, personality disorders, autism, and Asperger's disease.

16. The method of treating a mitochondrial dysfunctional disorder of claim 13, wherein the mitochondrial dysfunction disorder is a neurodegenerative disorder.

17. The method of treating a mitochondrial dysfunctional disorder of claim 16, wherein the neurodegenerative disorder is selected from Friedrich's ataxia, human deafness dystonia, amyotrophic lateral sclerosis, Parkinson's disease, Alzheimer's disease, Huntington's disease, and bipolar disorder (BD).

18. The method of treating a mitochondrial dysfunctional disorder of claim 13, wherein the mitochondrial dysfunctional disorder is diabetes.

19. The method of treating a mitochondrial dysfunctional disorder of claim 18, wherein the mitochondrial dysfunctional disorder is juvenile diabetes.

20. The method of treating a mitochondrial dysfunctional disorder of claim 18, wherein the mitochondrial dysfunctional disorder is type II diabetes.

21. The method of treating a mitochondrial dysfunctional disorder of claim 13, wherein the mitochondrial dysfunctional disorder is mitochondrial toxicity associated with therapeutic agents.

22. The method of treating a mitochondrial dysfunctional disorder of claim 21, the therapeutic agent is selected from a reverse transcriptase inhibitor, a protease inhibitor, and an inhibitor of dihydroorotate dehydrogenase (DHOD).

23. The method of treating a mitochondrial dysfunctional disorder of claim 22, wherein the reverse transcriptase inhibitor is selected from azidothymidine (AZT), stavudine (D4T), zalcitabine (ddC), didanosine (DDI), fluoroiodoarauracil (FIAU), lamivudine (3TC), abacavir, and combinations thereof.

24. The method of treating a mitochondrial dysfunctional disorder of claim 22, wherein the protease inhibitor is selected from ritonavir, indinavir, saquinavir, nelfinavir, and combinations thereof.

25. The method of treating a mitochondrial dysfunctional disorder of claim 22, wherein the inhibitor of dihydroorotate dehydrogenase (DHOD) is leflunomide, brequinar, or a combination thereof.

26. The method of treating a mitochondrial dysfunctional disorder of claim 13, wherein the mitochondrial dysfunctional disorder is a migraine.

27. The method of treating a mitochondrial dysfunctional disorder of claim 13, wherein the mitochondrial dysfunctional disorder is an ocular disorder associated with mitochondrial dysfunction.

28. The method of treating a mitochondrial dysfunctional disorder of claim 13, wherein the ocular disorder associated with mitochondrial dysfunction is selected from glaucoma, diabetic retinopathy, and age-related macular degeneration.

29. The method of treating a mitochondrial dysfunctional disorder of claim 13, wherein the mitochondrial dysfunctional disorder is an ischemia related condition.

30. The method of treating a mitochondrial dysfunctional disorder of claim 29, wherein the ischemic related condition is selected from angina, stroke, peripheral artery disease, and mesenteric ischemia.

31. A method of increasing muscle performance, wherein the method comprises administering a mitochondrial-protein complex comprising:
isolated mitochondria; and
transferrin-poly-L-lysine (Tf-PL) conjugate,

to a subject in need thereof.

32. The method of increasing muscle performance of claim 31, wherein the method comprises improving the subject's physical endurance.

33. The method of increasing muscle performance of claim 31, wherein the method comprises inhibiting the subject's physical fatigue.

34. The method of increasing muscle performance of claim 31, wherein the method comprises enhancing the subject's blood oxygen levels.

35. The method of increasing muscle performance of claim 31, wherein the method comprises increasing the subject's muscle ATP levels.

36. The method of increasing muscle performance of claim 31, wherein the method comprises reducing the level of lactic acid in the subject's blood.

37. A composition comprising a mitochondrial-protein complex and an endosomolytic escape protein, wherein the mitochondrial-protein complex comprises:
isolated mitochondria; and
asialoorosomucoid-poly-L-lysine (AsOR-PL) conjugate or transferrin-poly-L-lysine (Tf-PL) conjugate.

38. The composition of claim 37, wherein the endosomolytic escape protein is prepared by linking listeriolysin O (LLO) to AsOR.

39. The composition of claim 38, wherein the molar ratio of listeriolysin O (LLO) to AsOR is 2:1.

40. The composition of any one of claims 37-39, wherein the composition further comprises listeriolysin or another targetable endosomolytic agent.

41. The composition of any one of claims 37-40, wherein the endosomolytic escape protein is prepared by conjugating a pore-forming peptide to a carrier protein.

42. The composition of claim 41, wherein the pore-forming peptide is a bacterial, viral, or synthetic endosomolytic agent.

43. The composition of claim 41 or 42, wherein the carrier protein is AsOR.

44. A method of treating a liver disease, wherein the method comprises administering a mitochondrial-protein complex and AsOR-listeriolysin (LLO) to a subject in need thereof, wherein the mitochondrial-protein complex comprises:
isolated mitochondria; and
asialoorosomucoid-poly-L-lysine (AsOR-PL) conjugate.

45. The method of claim 44, wherein the liver disease is a liver disease of compromised mitochondrial function.

46. The method of claim 44 or 45, wherein the liver disease is selected from liver steatosis, nonalcoholic steatohepatitis (NASH), fibrosis, cirrhosis, alcoholic liver disease (ALD), liver cell lysis, cholestasis, steatosis, steatohepatitis, cirrhosis, liver cancer, viral hepatitis, and hepatocellular carcinoma (HCC).

47. The method of any one of claims 44-46, wherein the mitochondrial-protein complex and AsOR-listeriolysin (LLO) are administered to the subject by infusion.

48. A method of preparing a mitochondrial-protein complex comprising mixing
isolated mitochondria; and
asialoorosomucoid-poly-L-lysine (AsOR-PL) conjugate or transferrin-poly-L-lysine (Tf-PL) conjugate.

49. The method of preparing a mitochondrial-protein complex of claim 48, having a mean particle diameter of about 500 nm.

50. The method of preparing a mitochondrial-protein complex of claim 48 or 49, wherein the isolated mitochondria are prepared by a method comprising:
centrifugation of cells at lower speed;
centrifugation of cells at higher speed; and
filtration of cells through a membrane.

51. The method of preparing a mitochondrial-protein complex of claim 50, wherein centrifugation at lower speed comprises centrifuging the mitochondria at 1,000×g.

52. The method of preparing a mitochondrial-protein complex of claim 50 or 51, wherein centrifugation at higher speed comprises centrifuging the mitochondria at 7,000×g.

53. The method of preparing a mitochondrial-protein complex of any one of claims 50-52, wherein the membrane is a 1.2μ membrane filter.

54. The method of preparing a mitochondrial-protein complex of any one of claims 50-52, wherein the membrane is a 0.8μ membrane filter.

55. The method of preparing a mitochondrial-protein complex of any one of claims 50-52, wherein the method comprises a two-step filtration process.

56. The method of preparing a mitochondrial-protein complex of claim 55, wherein the mitochondria first pass through a 1.2μ membrane filter, then, in a second filtration step, the mitochondria pass through a 0.8μ membrane filter.

57. The method of preparing a mitochondrial-protein complex of any one of claims 48-56, wherein the method further comprises rupturing mammalian cells.

58. The method of preparing a mitochondrial-protein complex of claim 57, wherein the mammalian cells are liver cells.

59. The method of preparing a mitochondrial-protein complex of claim 57 or 58, wherein the mammalian cells are selected from human cells, porcine cells, canine cells, rodent cells, and feline cells.

60. The method of preparing a mitochondrial-protein complex of any one of claims 47-59, wherein the mammalian cells are rodent cells.

61. The method of preparing a mitochondrial-protein complex of any one of claims 57-60, wherein the mammalian cells are rat cells or mouse cells.

### Methods of Isolating Functional Mitochondria

Mitochondria include the mitochondrial genome, which is a circular double-stranded molecule, consisting of 16,569 base pairs. It contains 37 genes including 13 protein-encoding genes, 22 transfer RNA (tRNA) genes and two ribosomal RNA (rRNA) genes. The 13 protein-encoding genes are components of the mitochondrial respiratory chain. The wild type (wt)-wtDNA molecule may also include sequence polymorphism, but it remains fully functional. Structurally, mitochondria organelles range in diameter or width from 0.5 μm to 1 μm and have four compartments: the outer membrane, the inner membrane, the intermembrane space, and the matrix.

Disclosed herein are methods and reagents to isolate normal mitochondria from mammalian cells in a manner that results organelles with decreased mean particle diameters reflecting elimination of large impurities and have a high degree of outer membrane integrity and function. Currently available mitochondrial isolation kits result in organelles that have a large mean diameter, >700 nm. In addition, the yields are typically poor, and many of the mitochondria obtained are damaged. Mitochondrial diameter is important because lung capillaries have a diameter of about 1000 nm. Particles this size or larger can become trapped, blocking blood flow to the lungs, which can be lethal.

Furthermore, the diameter of pores in the lining of blood vessels through which the surfaces of liver cells have direct contact with blood traversing the liver have a diameter of about 100 nm. Particles that permit direct contact with liver cells can be bound by cell surface receptors.

Disclosed herein are methods for isolating functional mitochondria from mammalian cells comprising a combination of centrifugation and filtration. The disclosed methods produce mitochondria that have decreased mean particle diameters (e.g., less than 550 nm), are >95% intact, and highly pure (i.e., have increased mitochondrial DNA and decreased genomic contamination).

According to some embodiments, the term “functional mitochondria” refers to mitochondria that produce energy by consuming oxygen. According to some embodiments, functional mitochondria are intact mitochondria. In another embodiment, functional mitochondria consume oxygen at an increasing rate over time.

According to another embodiment, functional mitochondria have an intact outer membrane. As used herein, the term “intact mitochondria” refers to mitochondria comprising an outer and an inner membrane, an inter-membrane space, the cristae (formed by the inner membrane) and the matrix. As used herein, the term “a mitochondrial membrane” refers to the mitochondrial inner membrane, the mitochondrial outer membrane, or a combination thereof.

In another embodiment, intact mitochondria comprise mitochondrial DNA.

The key steps when isolating mitochondria from any tissue or cell are typically: (i) rupturing of cells by mechanical and/or chemical means, (ii) differential centrifugation at low speed to remove debris and extremely large cellular organelles, and (iii) centrifugation at a higher speed to isolate and collect and substantially pure mitochondria. The disclosed methods provide for subsequent filtration through restrictive membrane after centrifugation. In some embodiments, a 1.2μ membrane filter is used. In other embodiments, a 0.8μ is used. In yet other embodiments, the method provides for a two-step filtration process. For example, in some embodiments, the mitochondria first pass through a 1.2μ membrane filter, then, in a second filtration step, the mitochondria pass through a 0.8μ membrane filter.

In another embodiment, the functionality of mitochondria is measured by oxygen consumption. In another embodiment, oxygen consumption of mitochondria may be measured by any method known in the art such as, but not limited to, a Seahorse oxygen consumption analyzer.

Mitochondria integrity can be tested by screening for cytochrome c, porin, or cyclophilin D in the isolated mitochondria versus in the supernatant fraction (i.e., using commercially available antibody kits.

According to some embodiments, the isolated mitochondria disclosed herein are substantially free of intact cells. According to other embodiments, the isolated mitochondria disclosed herein is substantially free of mitochondrial clumps or aggregates or cellular debris or components larger than 5 μm. According to other embodiments, the isolated mitochondria disclosed herein is substantially devoid of particulate matter greater than 5 μm. As used herein, the term “particulate matter” refers to intact cells, cell debris, aggregates of mitochondria, aggregates of cellular debris, or a combination thereof. As used herein, a composition substantially devoid of exogenous particulate matter greater than 5 μm.

In some embodiments, the isolated mitochondria have a mean particle diameter of about 400 nm to about 550 nm, such as about 410 nm to about 550 nm, about 420 nm to about 550 nm, about 430 nm to about 550 nm, about 440 nm to about 550 nm, about 440 nm to about 540 nm, or about 410 nm to about 540 nm. For example, in some embodiments, the isolated mitochondria have a mean particle diameter of about 400 nm, 402 nm, 404 nm, 406 nm, 408 nm, 410 nm, 412 nm, 414 nm, 416 nm, 418 nm, 420 nm, 422 nm, 424 nm, 426 nm, 428 nm, 430 nm, 432 nm, 434 nm, 436 nm, 438 nm, 440 nm, 442 nm, 444 nm, 446 nm, 448 nm, 450 nm, 452 nm, 454 nm, 456 nm, 458 nm, 460 nm, 462 nm, 464 nm, 466 nm, 468 nm, 470 nm, 472 nm, 474 nm, 476 nm, 478 nm, 480 nm, 482 nm, 484 nm, 486 nm, 488 nm, 490 nm, 492 nm, 494 nm, 496 nm, 498 nm, about 510 nm, about 512 nm, about 514 nm, about 516 nm, about 518 nm, about 520 nm, about 522 nm, about 524 nm, about 526 nm, about 528 nm, about 530 nm, about 536, nm, about 538 nm, about 540 nm, about 542 nm, 544 nm, about 546 nm, about 548 nm, or about 550 nm. In some embodiments, the isolated mitochondria have a mean diameter of about 520 nm. In other embodiments, the isolated mitochondria have a mean particle diameter of about 536 nm. In yet other embodiments, the isolated mitochondria have a mean particle diameter of about 444 nm.

The isolated mitochondria for use in the methods, kits, and compositions of the invention can be obtained from any allogeneic, syngeneic, or xenogeneic source. In some embodiments, the mitochondria are extracted from mammalian cells. In preferred embodiments, the mammalian cells are mammalian liver cells. Exemplary sources of mammalian liver cells include those derived from mammals including humans as well as other animals such as pigs, dogs, rodents, and felines (i.e., human liver cells, porcine liver cells, canine liver cells, rodent liver cells, and feline liver cells). In some embodiments, the mammalian liver cells are rat liver cells. In other embodiments, the mammalian liver cells are mouse liver cells.

The source of cells for mitochondrial isolation can be the patient to be treated himself or herself, a relative, an unrelated donor or a donor of another species. Once isolated, mitochondria can be cultured by propagation within donor cells, and these cultured mitochondria can also be used in the mitochondrial replacement therapy described herein.

Other organs that may be used as sources of mitochondria include, but are not limited to, rapidly proliferating cells (e.g., mucosa, skin, bone marrow), metabolically active tissues (e.g., skeletal muscle, cardiac muscle, white blood cell precursors, and red blood cell precursors), and progenitor cells. Sources of mitochondria also may be tissues from an individual person or cells in culture.

### Mitochondrial-Protein Complexes

In some embodiments, a mitochondrial-protein complex was prepared from the isolated mitochondria disclosed herein and asialoorosomucoid-poly-L-lysine (AsOR-PL) conjugate or transferrin-poly-1-lysine (Tf-PL) conjugate. In some embodiments, the mitochondrial-protein complex has a mean particle diameter of about 450 nm to about 510 nm, such as about 455 nm to about 510 nm, about 460 nm to about 510 nm, about 465 nm to about 510 nm, about 470 nm to about 510 nm, about 475 nm to about 510 nm, about 480 nm to about 510 nm, about 485 nm to about 510 nm, about 485 nm to about 510 nm, about 490 nm to about 510 nm, about 495 nm to about 510 nm, or about 495 nm to about 505. nm. For example, in some embodiments, the mitochondrial-protein complex has a mean particle diameter of about 450 nm, about 455 nm, about 460 nm, about 465 nm, about 470 nm, about 475 nm, about 480 nm, about 485 nm, about 490 nm, about 495 nm, about 500 nm, about 505 nm, or about 510 nm. In certain embodiments, the mitochondrial-protein complex has a mean particle diameter of about 500 nm.

The disclosed mitochondrial-protein complexes are highly useful in a broad range of applications. In some embodiments, the disclosed mitochondrial-protein complexes may be used in a method for transplanting mitochondria into a subject in need thereof. In some embodiments, the disclosed mitochondrial-protein complexes may be used in a method of treating a mitochondrial dysfunctional disorder. In some embodiments, the disclosed mitochondrial-protein complexes may be used in a method for increasing muscle performance. In some embodiments, the disclosed mitochondrial-protein complexes may be used in a method for treating liver disease.

### Compositions and Pharmaceutical Compositions

The present invention provides a pharmaceutical composition comprising the functional mitochondria isolated by the methods disclosed herein. In some embodiments, the pharmaceutical composition comprises functional mitochondria with an intact outer membrane.

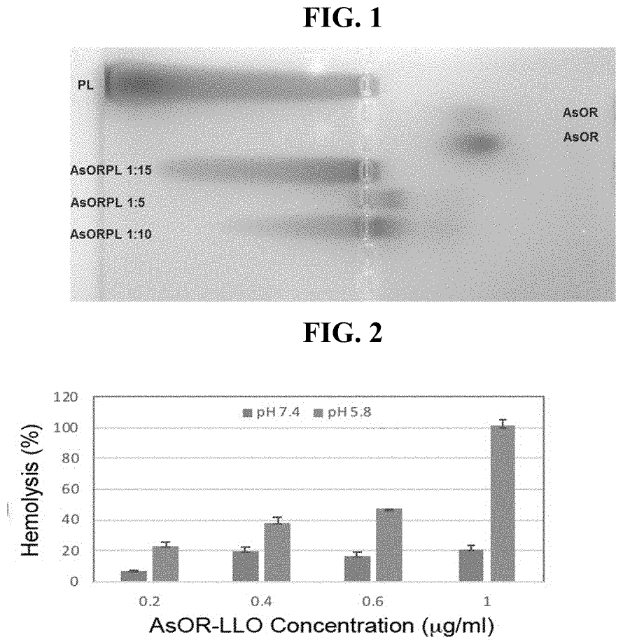

In some embodiments, disclosed herein are compositions and methods that are designed to take advantage of asialoglycoprotein receptor on the surface of mammalian liver cells, which efficiently binds and internalizes galactose-terminal (asialo-) glycoproteins within endosomes ultimately degrading its contents by fusion with lysosomes. In some embodiments, an asialoglycoprotein (the asialooromucoid (AsOR)) is linked to a polymer of lysine (i.e., polylysine (PL)), which converts the asialoglycoprotein into a highly positively charged conjugate (i.e., AsOR-PL). Mixing of the AsOR-PL conjugate with mitochondria, which are negatively changed, affords a stable AsOR-PL mitochondrial complex due to electrostatic binding. To allow internalized mitochondria to escape endosomes, an endosomolytic agent, listeriolysin O (LLO) was linked to AsOR to form a targetable endosomolytic agent, AsOR-LLO. Co-internalization of complexed mitochondria with a targetable endosomolytic agent, (FIG. 1). AsOR-LLO, resulted in release of internalized mitochondria (FIG. 2).

In some embodiments, disclosed herein is a composition a mitochondrial-protein complex and a targetable endosomolytic escape protein, wherein the mitochondrial-protein complex comprises isolated mitochondria; and asialoorosomucoid-poly-l-lysine (AsOR-PL) conjugate or transferrin-poly-L-lysine (Tf-PL) conjugate. In some embodiments, the endosomolytic escape protein is prepared by linking listeriolysin O (LLO) to AsOR. In certain embodiments, the molar ratio of listeriolysin O (LLO) to AsOR is 2:1. In some embodiments, the composition further comprises listeriolysin or another targetable endosomolytic agent. In some embodiments, the endosomolytic escape protein is prepared by conjugating a pore-forming peptide to a carrier protein. In certain embodiments, the pore-forming peptide is a bacterial, viral, or synthetic endosomolytic agent. In certain other embodiments, the carrier protein is AsOR.

In some embodiments, the compositions and pharmaceutical compositions disclosed herein comprise functional mitochondria with a mean particle diameter of about 400 nm to about 550 nm, such as about 410 nm to about 550 nm, about 420 nm to about 550 nm, about 430 nm to about 550 nm, about 440 nm to about 550 nm, about 440 nm to about 540 nm, or about 410 nm to about 540 nm. For example, in some embodiments, the pharmaceutical composition comprises functional mitochondria with a mean particle diameter of about 510 nm, about 512 nm, about 514 nm, about 516 nm, about 518 nm, about 520 nm, about 522 nm, about 524 nm, about 526 nm, about 528 nm, about 530 nm, about 536, nm, about 538 nm, about 540 nm, about 542 nm, 544 nm, about 546 nm, about 548 nm, or about 550 nm. In one embodiment, the pharmaceutical composition comprises functional mitochondria with a mean diameter of about 520 nm. In another embodiment, the pharmaceutical composition comprises functional mitochondria with a mean particle diameter of about 536 nm.

In some embodiments, the compositions and pharmaceutical compositions disclosed herein comprise a mitochondrial-protein complex with a mean particle diameter of about 450 nm to about 510 nm, such as about 455 nm to about 510 nm, about 460 nm to about 510 nm, about 465 nm to about 510 nm, about 470 nm to about 510 nm, about 475 nm to about 510 nm, about 480 nm to about 510 nm, about 485 nm to about 510 nm, about 485 nm to about 510 nm, about 490 nm to about 510 nm, about 495 nm to about 510 nm, or about 495 nm to about 505. nm. For example, in some embodiments, the pharmaceutical composition comprises a mitochondrial-protein complex with a mean particle diameter of about 450 nm, about 455 nm, about 460 nm, about 465 nm, about 470 nm, about 475 nm, about 480 nm, about 485 nm, about 490 nm, about 495 nm, about 500 nm, about 505 nm, or about 510 nm. In certain embodiments, the pharmaceutical composition comprises a mitochondrial-protein complex with a mean particle diameter of about 500 nm.

In some embodiments, the disclosed compositions and/or pharmaceutical compositions may be used in a method for transplanting mitochondria into a subject in need thereof. In some embodiments, the disclosed pharmaceutical compositions may be used in a method of treating a mitochondrial dysfunctional disorder. In some embodiments, the disclosed pharmaceutical compositions may be used in a method for increasing muscle performance. In some embodiments, the disclosed pharmaceutical compositions may be used in a method for treating liver disease.

### Formulation

The functional mitochondria isolated by the methods disclosed herein may be prepared as a pharmaceutical formulation, containing one or more pharmaceutically acceptable excipients for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), See Remington's Pharmaceutical Sciences (18th Ed., A. R. Gennaro, ed., Mack Publishing Company 1990).

For example, lipids may be used in the present invention as a carrier. The lipids may be natural, synthetic or semisynthetic (i.e., modified natural). Lipids useful in formulating the compositions of the invention, without limitation, fatty acids, lysolipids, oils (including safflower, soybean and peanut oil), phosphatidylcholine with both saturated and unsaturated lipids, including phosphatidylcholine; dioleoylphosphatidylcholine; dimyristoylphosphatidylcholine; dipentadecanoylphosphatidylcholine, dilauroylphosphatidylcholine, dioleoylphosphatidylcholine, dipalmitoylphosphatidylcholine; and distearoylphosphatidylcholine; phosphatidylethanolamines, such as dioleoylphosphatidylethanolamine; phosphatidylserine; phosphatidylglycerol; phosphatidylinositol, sphingolipids, such as sphingomyelin; glycolipids, such as ganglioside GM1 and GM2; glucolipids; sulfatides; glycosphingolipids; phosphatidic acid; palmitic acid; stearic acid; arachidonic acid; oleic acid; lipids bearing polymers such as polyethyleneglycol, chitin, hyaluronic acid or polyvinylpyrrolidone; lipids bearing sulfonated mono-, di-, oligo- or polysaccharides; cholesterol, cholesterol sulfate and cholesterol hemisuccinate; tocopherol hemisuccinate, lipids with ether and ester-linked fatty acids, polymerized lipids (a wide variety of which are known in the art), diacetyl phosphate, stearylamine, cardiolipin, phospholipids with short chain fatty acids of about 6 to about 8 carbons in length, synthetic phospholipids with asymmetric acyl chains (e.g., with one acyl chain of about 6 carbons and another acyl chain of about 12 carbons), 6-(5-cholesten-3b-yloxy)-1-thio-β-D-galactopyranoside, digalactosyldiglyceride, 6-(5-cholesten-3.beta.-yloxy)hexyl-6-amino-6-deoxy-1-thio-β-D-galacto pyranoside, 6-(5-cholesten-3.beta.-yloxy)hexyl-6-amino-6-deoxyl-1-thio-.alpha.-D-manno pyranoside, 12-(((7′-diethylamino-coumarin-3-yl)carbonyl)methylamino)-octadecanoic acid; N-[12-(((7′-diethylaminocoumarin-3-yl)carbonyl)methyl-amino)octadecanoyl]-2-aminopalmitic acid; (cholesteryl)4′-trimethyl-ammonio)butanoate; N-succinyldioleoylphosphatidylethanol amine; 1,2-dioleoyl-sn-glycerol; 1,2-dipalmitoyl-3-succinyl-glycerol; 1,3-dipalmitoyl-2-succinylglycerol; 1-hexadecyl-2-palmitoyl-glycerophosphoethanolamine; palmitoylhomocysteine; and combinations thereof. Vesicles or other structures may be formed of the lipids, either as monolayers, bilayers, or multilayers and may or may not have a further coating. Vesicles or other lipid structures used as carriers can further include, e.g., peptides, polypeptides, glycoproteins, or other constituents useful for the generation, viability, or targeting of such carriers.

Cationic lipids and other derivatized lipids and lipid mixtures also may be useful as carriers for use in the methods, kits, and compositions of the invention. Suitable cationic lipids include dimyristyl oxypropyl-3-dimethylhydroxy ethylammonium bromide (DMRIE), dilauryl oxypropyl-3-dimethylhydroxy ethylammonium bromide (DLRIE), N-[1-(2,3-dioleoyloxyl) propal]-n,n,n-trimethylammonium sulfate (DOTAP), dioleoylphosphatidylethanolamine (DOPE), dipalmitoylethylphosphatidylcholine (DPEPC), dioleoylphosphatidylcholine (DOPC), polylysine, lipopolylysine, didoceyl methylammonium bromide (DDAB), 2,3-dioleoyloxy-N-[2-(sperminecarboxamidoethyl]-N,N-di-methyl-1-propanamin ium trifluoroacetate (DOSPA), cetyltrimethylammonium bromide (CTAB), lysyl-PE, 3.beta.-[N,(N′,N′-dimethylaminoethane)-carbamoyl]cholesterol (DC-Cholesterol, also known as DC-Chol), (-alanyl cholesterol, N-[1-(2,3-dioleoyloxy)propyl]-N,N,N-trimethylammonium chloride (DOTMA), dipalmitoylphosphatidylethanolamine-5-carboxyspermylamide (DPPES), dicaproylphosphatidylethanolamine (DCPE), 4-dimethylaminopyridine (DMAP), dimyristoylphosphatidylethanolamine (DMPE), dioleoylethylphosphocholine (DOEPC), dioctadecylamidoglycyl spermidine (DOGS), N-[1-(2,3-dioleoyloxy)propyl]-N-[1-(2-hydroxyethyl)]-N,N-dimethylammonium iodide (DOHME), Lipofectin (DOTMA+DOPE, Life Technologies, Inc., Gaithersburg, Md.), Lipofectamine (DOSPA+DOPE, Life Technologies, Inc., Gaithersburg, Md.), Transfectace (Life Technologies, Inc., Gaithersburg, Md.), Transfectam (Promega Ltd., Madison, Wis.), Cytofectin (Life Technologies Inc., Gaithersburg, Md.). Other representative cationic lipids include but are not limited to phosphatidylethanolamine, phospatidylcholine, glycero-3-ethylphosphatidylcholine and fatty acyl esters thereof, di- and trimethyl ammonium propane, di- and tri-ethylammonium propane and fatty acyl esters thereof.

Additionally, a wide array of synthetic cationic lipids function as compounds useful in the invention. These include common natural lipids derivatized to contain one or more basic functional groups. Examples of lipids that may be so modified include but are not limited to dimethyldioctadecylammonium bromide, sphingolipids, sphingomyelin, lysolipids, glycolipids such as ganglioside GMI, sulfatides, glycosphingolipids, cholesterol and cholesterol esters and salts, N-succinyldioleoylphosphatidylethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl-ethanolamine and palmitoylhomocystiene.

The methods, kits, and compositions of the invention can include the formulation of mitochondria with one or more agents (e.g., vitamins, antioxidants, acetyl-L-carnitine, alpha-lipoic acid, cardiolipin, fatty acids, lithium carbonate, lithium citrate, calcium, or s-adenosyl-L-methionine) or mixtures thereof, such as those described herein.

The mitochondria for use in the methods, compositions, and kits of the invention can be packaged in unit dosage forms, for example in vials, ampoules, pre-filled syringes, or sachets.

### Administration

Formulations comprising functional mitochondria prepared by the methods disclosed herein can be administered to subjects in therapeutically effective amounts. The preferred dosage to be administered is likely to depend on such variables as the type and extent of the disorder, the overall health status of the particular subject, the specific compound being administered, the excipients used to formulate the mitochondria, and its route of administration. Standard clinical trials maybe used to optimize the dose and dosing frequency for any condition and route of administration.

For systemic administration, mitochondria can be, without limitation, administered by intranasal, intravenous, intra-arterial, subcutaneous, or intramuscular routes. In preferred embodiments, the mitochondria are administered intravenously. The mitochondria can be administered alone (e.g., as a monotherapy), after pretreatment with one of several second agents described herein, or in combination with one of several second agents described herein (e.g., either formulated together and administered simultaneously, or formulated separately and administered within two (2) hours of each other).

For the treatment of conditions associated with localized mitochondrial dysfunction (e.g., ocular disorders, neurodegenerative disorders, neuropsychiatric disorders, and other localized tissues) it may be desirable to administer the isolated and substantially pure mitochondria locally. Local routes of administration include, without limitation, local injection, intracranial, intracerebroventricular, intracerebral, intraorbital, ophthalmic, intraventricular, intracapsular, intraspinal, intrathecal, intracisternal, intraperitoneal, intranasal, or topical administration. For example, the functional mitochondria prepared by the methods disclosed herein can be administered directly into discrete areas or nuclei of the brain, e.g., the rostral ventromedial medulla (RVM) or a brain ventricle, or onto the dura mater.

### Combination Therapy

The functional mitochondria prepared by the methods disclosed herein can be used in combination with a second agent. In some embodiments, the second agent is selected from vitamins (e.g., vitamin A, vitamin C, vitamin D, vitamin E, vitamin K, folic acid, choline, vitamin B₁, vitamin B₂, vitamin B₅, vitamin B₆, and vitamin B₁₂, biotin, nicotinamide), antioxidants (e.g., betacarotene, coenzyme Q, selenium, superoxide dismutase, glutathione peroxide, uridine, catalase, creatine succinate, pyruvate, dihydroxyacetone), acetyl-L-carnitine, alpha-lipoic acid, cardiolipin, fatty acids (e.g., omega-3 fatty acids or omega-6 fatty acids), lithium carbonate, lithium citrate, calcium, and s-adenosyl-L-methionine. These additional agents can be directly administered to the patient (e.g., orally or intravenously) and formulated together with the isolated and substantially pure mitochondria or formulated and administered separately.

Conditions Associated with Mitochondrial Dysfunction

Mitochondria are involved in a strikingly diverse range of disease processes. Primary genetic disorders fall into two broad classes: those with deficiencies in either nuclear or mitochondrial genes (see Sholte, J. Bioenerg. Biomembr. 20:161 (1988), reporting over 60 human diseases with defects in nuclear genes encoding mitochondrial functions). Mitochondrial dysfunction is also recognized as a contributor to common diseases with multi-factorial pathogenesis.

Conditions associated with mitochondrial dysfunction include those in which deficits in mitochondrial respiratory chain activity contribute to the development of pathophysiology of such diseases or disorders in a mammal. This includes (1) congenital genetic deficiencies in the activity of one or more components of the mitochondrial respiratory or electron transport chain; and (2) acquired deficiencies in the levels or activities of one or more components of the mitochondrial respiratory chain, wherein such deficiencies are caused by (a) oxidative damage during aging; (b) elevated intracellular calcium; (c) exposure of affected cells to nitric oxide; (d) hypoxia or ischemia; (e) microtubule-associated deficits in axonal transport of mitochondria, or (f) expression of mitochondrial uncoupling proteins.

Common symptoms of mitochondrial dysfunction include cardiomyopathy, muscle weakness and atrophy, developmental delays (involving motor, language, cognitive or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

### Neuropsychiatric Disorders

The brain requires tenfold the energy on average of the rest of the body. Many neuropsychiatric disorders may be associated with abnormalities of energy production or mitochondrial dysfunction, in particular. Neuropsychiatric disorders include, without limitation bipolar disorder (BD), schizophrenia, depression, anxiety disorders, attention deficit disorders, addictive disorders, personality disorders, autism, and Asperger's disease. The methods, kits, and compositions of the invention can be used for the treatment of neuropsychiatric disorders.

### Neurodegenerative Disorders

The methods, kits, and compositions of the invention can be used for the treatment of neurodegenerative disorders. Many progressive neurological diseases result from the execution of neurons by mitochondrial apoptosis. Friedrich's ataxia results from a genetic defect in the frataxin gene, which is involved in mitochondrial iron transport (Babcock et al., Science 276:1709 (1997)); human deafness dystonia results from a defect in a small component of the mitochondrial protein import machinery (Koehler et al., Proc. Natl. Acad. Sci. USA 96:2141 (1999)); one well-characterized cause of amyotrophic lateral sclerosis is deficiency in Cu—Zn superoxide dismutase, which is located in the mitochondrial intermembrane space as well as the cytoplasm (Deng et al. Science 261:1047 (1993)). The discovery that several environmental toxins cause Parkinsonism by inhibiting respiratory complex I and promoting the generation of reactive oxygen species has made this complex a focus for research on the basis of Parkinson's disease (Dawson et al., Science 302:819 (2003)). More recently, the mitochondrial protein encoded by PINK 1 has provided a direct link between mitochondria and Parkinson's disease (Valente et al., Science 304:1158 (2004)). Alzheimer's disease is also linked to mitochondrial toxicity through the mitochondrial protein ABAD, a target of amyloid (Lustbader et al., Science 304:448 (2004)). Huntington's Disease has been associated with defects in energy metabolism that appear to be widespread, affecting both the brain and peripheral tissues, and arising from mitochondrial dysfunction (Leegwater-Kim et al., NeuroRx 1:128 (2004)). A basic abnormality involved in the pathogenesis of bipolar disorder (BD) is believed to involve energy production and in particular, mitochondrial activity. Evidence from many sources, including, postmortem, genetic, brain imaging and peripheral cell studies support energy deficits and mitochondrial dysfunction as one important causative factor in the development of BD (see Hough et al., Bipolar Disord. 2:145 (2000), Fattal et al., Psychosomatics 47:1 (2006), and Kato et al., Bipolar Disord. 2:180 (2000)).

### Diabetes and Metabolic Disease

The methods, kits, and compositions of the invention can be used for the treatment of diabetes and metabolic disease. The central role of mitochondria in metabolism of carbohydrates and fatty acids gives this organelle an important function in diabetes (Maechler et al., Nature 414:807 (2001)). A mouse knockout of an abundant mitochondrial transcription factor has provided a model for 13-cell ablation in juvenile diabetes (Silva et al., Nat. Genet. 26:335 (2000)). Mutations in mtDNA and in PPARγ, a master regulator of mitochondrial biogenesis, are correlated with type II diabetes. Insulin release depends on mitochondrial function as influenced by the expression of the membrane transporter UCP2 (Petersen et al., Science 300:1140 (2003); Zhang et al., Cell 105:745 (2001)). The activity of thiazolidinediones as antidiabetic agents appears to depend on their ability to serve as ligands for PPARγ and its co-activator, PGC-1, in their control of expression of nuclear genes for mitochondrial gene products (Mootha et al., Nature Genet 34:267 (2003); Puigserver et al., Endocr. Rev. 24:78 (2003)).

### Mitochondrial Toxicity of Therapeutic Agents

The methods, kits, and compositions of the invention can be used for the treatment of toxicity associated with therapeutic agents. The past few decades have witnessed significant progress in development of chemotherapeutic agents for cancer and viral diseases. In the case of conventional cancer chemotherapy, the goal of selectively killing tumor cells has been difficult to attain due to collateral toxicity to normal cells. Cancer chemotherapeutic agents delivered to damage nuclear DNA also directly damage mtDNA as well, even in “resting tissues” where nuclear DNA replication is inactive, but mtDNA replication continues. Mitochondria are poorly equipped to repair this sort of collateral damage (Bhatia et al., Nature Reviews Cancer 2:124 (2002)). Nucleoside analogues used as either anticancer or antiviral agents can also have significant mitochondrial toxicity. The best-known examples include the inhibition of DNA polymerase y by AZT and dideoxynucleosides used to target the related HIV reverse transcriptase and the fatal hepatotoxicity of fialuridine observed when this agent was tested for activity against hepatitis B virus (Lewis et al., Nat. Med. 1:417 (1995)). In addition, the myopathy and rhabdomyolysis associated with the popular cholesterol-lowering statins (Thompson et al., JAMA 289:1681 (2003)) are believed to involve interference with mitochondrial ubiquinone biosynthesis. Accordingly, the methods, kits, and compositions of the invention can be used to ameliorate the toxicity of drugs. Pharmaceutical agents associated with mitochondrial toxicity include reverse transcriptase inhibitors (e.g., azidothymidine (AZT), stavudine (D4T), zalcitabine (ddC), didanosine (DDI), fluoroiodoarauracil (FIAU), lamivudine (3TC), abacavir), protease inhibitors (e.g., ritonavir, indinavir, saquinavir, nelfinavir), and inhibitors of dihydroorotate dehydrogenase (DHOD) (e.g., leflunomide, brequinar), among others. The methods of the present invention can be effective in the treatment of mitochondrial-related drug toxicity of the liver.

### Liver Disease

Mitochondria play an important role in liver cell metabolism, are the main sites of fatty acid oxidation and oxidative phosphorylation, play a major role in cell redox homeostasis, and maintain normal liver function (D. Degli Esposti, J. Hamelin, N. Bosselut et al., “Mitochondrial roles and cytoprotection in chronic liver injury,” Biochem Res Int. 2012; 2012:387626). Therefore, the methods, kits, and compositions of the invention can be used for the treatment of a liver disease. In some embodiments, the liver disease is a liver disease of compromised mitochondrial function, such as, for example, liver steatosis, nonalcoholic steatohepatitis (NASH), fibrosis, cirrhosis, alcoholic liver disease (ALD), liver cell lysis, cholestasis, steatosis, steatohepatitis, cirrhosis, liver cancer, viral hepatitis, and hepatocellular carcinoma (HCC). The methods of the present invention can be effective in the treatment of mitochondrial-related pathological conditions of the liver.

### Migraine

The methods, kits, and compositions of the invention can be used for the treatment of migraine. Metabolic studies on patients with recurrent migraine headaches indicate that deficits in mitochondrial activity are commonly associated with this disorder, manifesting as impaired-oxidative phosphorylation and excess lactate production. Such deficits are not necessarily due to genetic defects in mitochondrial DNA. Migraines are hypersensitive to nitric oxide, an endogenous inhibitor of cytochrome c oxidase. In addition, patients with mitochondrial cytopathies, e.g., MELAS, often have recurrent migraines.

Ocular Disorders Associated with Mitochondrial Dysfunction

The methods, kits, and compositions of the invention can be used for the treatment of ocular disorders, such as glaucoma, diabetic retinopathy, and age-related macular degeneration. Retinal damage is attributed to free radical initiated reactions in glaucoma, diabetic retinopathy and age-related macular degeneration (AMD). The eye is a part of the central nervous system and has limited regenerative capability. The retina is composed of numerous nerve cells which contain the highest concentration of polyunsaturated fatty acids (PFA) and subject to oxidation. Free radicals are generated by UV light entering the eye and mitochondria in the rods and cones, which generate the energy necessary to transform light into visual impulses. Free radicals cause peroxidation of the PFA by hydroxyl or superoxide radicals which in turn propagate additional free radicals. The free radicals cause temporary or permanent damage to retinal tissue.

Glaucoma is usually viewed as a disorder that causes an elevated intraocular pressure (TOP) that results in permanent damage to the retinal nerve fibers, but a sixth of all glaucoma cases do not develop an elevated IOP. This disorder is now perceived as one of reduced vascular perfusion and an increase in neurotoxic factors. Recent studies have implicated elevated levels of glutamate, nitric oxide and peroxynitirite in the eye as the causes of the death of retinal ganglion cells.

Diabetic retinopathy occurs when the underlying blood vessels develop microvascular abnormalities consisting primarily of microaneurysms and intraretinal hemorrhages. Oxidative metabolites are directly involved with the pathogenesis of diabetic retinopathy and free radicals augment the generation of growth factors that lead to enhanced proliferative activity. Nitric oxide produced by endothelial cells of the vessels may also cause smooth muscle cells to relax and result in vasodilation of segments of the vessel. Ischemia and hypoxia of the retina occur after thickening of the arterial basement membrane, endothelial proliferation and loss of pericytes. The inadequate oxygenation causes capillary obliteration or nonperfusion, arteriolar-venular shunts, sluggish blood flow and an impaired ability of RBCs to release oxygen. Lipid peroxidation of the retinal tissues also occurs as a result of free radical damage. The methods of the present invention can be effective in the treatment of mitochondrial-related pathological conditions of the eye.

### Ischemia-Related Conditions

The methods, kits, and compositions of the invention can be used for the treatment of ischemia related conditions (e.g., angina, stroke, peripheral artery disease, mesenteric ischemia). Oxygen deficiency results in both direct inhibition of mitochondrial respiratory chain activity by depriving cells of a terminal electron acceptor for cytochrome c reoxidation at Complex IV, and indirectly, especially in the nervous system, via secondary post-anoxic excitotoxicity and nitric oxide formation. In conditions like cerebral anoxia, angina, or sickle cell anemia crises, tissues are relatively hypoxic. In such cases, an increase in mitochondrial activity provides protection of affected tissues from deleterious effects of hypoxia, attenuate secondary delayed cell death, and accelerate recovery from hypoxic tissue stress and injury. The methods, kits, and compositions of the invention can be useful for preventing delayed cell death (apoptosis in regions like the hippocampus or cortex occurring about 2- to 5-days after an episode of cerebral ischemia) after ischemic or hypoxic insult, and other mitochondrial-related pathological conditions of brain.

to the heart, brain, legs, or intestines.

### Muscle Function

The methods, kits, and compositions of the invention can be used for enhancing muscle performance. For example, the methods, kits, and compositions of the invention may be useful for improving physical endurance (e.g., ability to perform a physical task such as exercise, physical labor, sports activities, etc.), inhibiting or retarding physical fatigue, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood.

Enhanced sports performance, strength, speed and endurance are typically measured by an increase in muscular contraction strength, increase in amplitude of muscle contraction, shortening of muscle reaction time between stimulation and contraction, the ability to overcome muscle fatigue, and ability to maintain activity for longer periods of time. Aside from muscle performance during endurance exercise, free radicals and oxidative stress parameters are affected in pathophysiological states. A substantial body of data now suggests that oxidative stress contributes to muscle wasting or atrophy in pathophysiological states (see Clarkson, Crit. Rev. Food Sci. Nutr. 35:31 (1995); and Powers et al., Proc. Nutr. Soc. 58:1025 (1999)). For example, in muscular dystrophies dystrophin-glycoprotein complex (DGC) defects suggest that one mechanism of cellular injury is functional ischemia related to alterations in cellular NOS and disruption of a normal protective action of NO. Rando (Microsc. Res. Tech. 55:223 (2001)) has shown that oxidative injury precedes pathologic changes and that muscle cells with defects in the DGC have an increased susceptibility to oxidant challenges. Excessive lipid peroxidation due to free radicals has also been shown to be a factor in myopathic diseases such as McArdle's disease (see Russo et al., Med. Hypotheses. 39:147 (1992)). Furthermore, mitochondrial dysfunction is a well-known correlate of age-related muscle wasting (sarcopenia) and free radical damage has been suggested, though poorly investigated, as a contributing factor (see Navarro et al., Front. Biosci. 6:D26 (2001)). Other indications include acute sarcopenia, for example muscle atrophy and/or cachexia associated with burns, bed rest, limb immobilization, or major thoracic, abdominal, and/or orthopedic surgery. The methods of the present invention can be effective in the treatment of mitochondrial-related pathological conditions of muscle.

### Aging

The methods, kits, and compositions of the invention can be used for the treatment of aging and conditions associated therewith. During normal aging, there is a progressive decline in mitochondrial respiratory chain function. Beginning about age 40, there is an exponential rise in accumulation of mitochondrial DNA defects in humans, and a concurrent decline in nuclear-regulated elements of mitochondrial respiratory activity. Many mitochondrial DNA lesions have a selection advantage during mitochondrial turnover, especially in post-mitotic cells. The proposed mechanism is that mitochondria with a defective respiratory chain produce less oxidative damage to themselves than do mitochondria with intact functional respiratory chains (mitochondrial respiration is the primary source of free radicals in the body). Therefore, normally functioning mitochondria accumulate oxidative damage to membrane lipids more rapidly than do defective mitochondria, and are, therefore, “tagged” for degradation by the autophagic and lysosomal systems. Since mitochondria within cells have a half-life of about 10 days, a selection advantage can result in rapid replacement of functional mitochondria with those with diminished respiratory activity, especially in slowly dividing cells. The net result is that once a mutation in a gene for a mitochondrial protein that reduces oxidative damage to mitochondria occurs, such defective mitochondria will rapidly populate the cell, diminishing or eliminating its respiratory capabilities. The inexorable decline of mitochondrial function with age contributes to the aging-related conditions of neurodegeneration, and type II diabetes. Just as oxidative stress underlies some of these defined diseases, it is thought to contribute to generalized aging (Harman, Proc. Natl. Acad. Sci. USA 78:7124 (1981)). Mutations in C. elegans and D. melanogaster that reduce mitochondrial oxidative stress have been shown to prolong lifespan in these organisms (Hekimi et al., Science 299:1351 (2003)). Moreover, mammals maintained on calorie-restricted diets have a reduced metabolic rate that is thought to contribute to significantly increased longevity. Numerous studies have documented an increase in point mutations and deletions in mtDNA with advancing age. Furthermore, Trifunovic et al. have recently showed that mice engineered to express an error prone mitochondrial DNA polymerase can serve as an excellent model for premature ageing (Nature 429:417 (2004)).

All references and publications included herein are incorporated by reference. The following examples are not intended to be limiting.

### EXEMPLIFICATION

### Example 1: Preparation of Uniform and Functional Mitochondria from Fresh Liver

This example provides a description of a two-step procedure consisting of sequential centrifugation followed by membrane filtration results in preparations of highly purified, uniform, and functional mitochondria, and unexpectedly significantly improves the preparation of mitochondria from fresh liver tissue.

### Methods

Mitochondrial Isolation from Mouse Liver

CD-1(ICR) mice of 21-30 days of age were anesthetized using ketamine/xylazine administered by intraperitoneal injection. The liver was removed and homogenized in the mitochondria isolation buffer (IB), as described by Frezza et al. (“Organelle isolation: functional mitochondria from mouse liver, muscle and cultured fibroblasts.” Nat Protoc 2007; 2:287-95) 100 mL of IB was prepared by adding 10 ml of 0.1 M Tris-MOPS and 1 ml of 0.1 M EGTA to 20 mL of 1M sucrose, adjusting the pH to 7.4 and bringing the volume to 100 mL with distilled water. Protease inhibitor was added in a 1:100 (volume) ratio to IB.

The homogenate was transferred to 15 mL conical centrifuge tubes (USA Scientific, Orlando Fla.) and centrifuged at 1000×g on an Allegra X-14R Centrifuge (Beckman Coulter, Indianapolis, Ind.) for 10 min at 4° C., to pellet the nuclei, debris, and large particles. The supernatant was subsequently transferred to 1.5 mL tubes (Eppendorf, San Diego, Calif.) and centrifuged at 7,000×g on an Eppendorf centrifuge 5424 R (Eppendorf) for 10 min at 4° C. The supernatant was discarded, the pellet was resuspended in 100 μL of ice-cold IB, and transferred to a new 1.5 mL tube, and brought to 1 mL with IB. The suspension was centrifuged at 7,000×g for 5 min at 4° C. to eliminate small particles and debris. The supernatant was discarded, and pellets were combined and resuspended in 1 mL of IB. A final purification step consisted of sequential passage of the resuspended mitochondria through 1.2μ and 0.8μ pore filters using a 3 mL syringe. The preparation kept on ice and used within 1-3 hours for measurement of mitochondrial DNA, size, charge, integrity, and function.

### Size Analysis

The size of isolated mitochondria was measured using a laser light scattering particle size analyzer (Model #90 Plus, Brookhaven Instruments, Holtsville, N.Y.). Freshly prepared mitochondria were suspended in IB, and particle size was measured.

Quantitative Real Time Polymerase Chain Reaction (qPCR)

DNA was extracted from isolated mitochondria (DNeasy™ kit, Qiagen, Netherlands). Mitochondrial DNA (mtDNA), 100 ng, was used as a template for qPCR with primers designed to amplify a specific region of mouse mitochondria 116 bp product length (primers; 5′-TCGCCTACTCCTCAGTTAGCCACA-3′ (SEQ ID NO: 1), 5′-TGATGATGTGAGGCCATGTGCGA-3′ (SEQ ID NO: 2), Integrated DNA Technology, Skokie, Ill.). ΔΔCt values were calculated to compare mtDNA to lactate dehydrogenase A (LDHA) signals as an indication of nuclear DNA contamination (primers; 5′-TAATGAAGGACTTGGCAGATGAACT-3′(SEQ ID NO: 3), 5′-ACGGCTTTCTCCCTCTTGCT-3′ (SEQ ID NO: 4), Integrated DNA Technology). Copy number was determined by preparation of a specific sequence of mouse mitochondrial DNA (5′-TCGCCTACTCCTCAGTTAGCCACATAGCAC TTGTTATTGCATCAATCATAATCCAAACTCCATGAAGCTTCATAGGAGCAACAATAC TAATAATCGCACATGGCCTCACATCATCA-3′ (SEQ ID NO: 5)) by amplification which was used to generate a standard curve from which quantities of experimental samples could be determined.

### Cytochrome C Assay

The integrity of mitochondrial outer membrane (MOM) was determined by cytochrome c oxidase assay kit (Sigma-Aldrich, St. Louis, Mo.) as instructed by the manufacturer. In brief, samples of freshly prepared mitochondria were suspended in 0.2 mg/mL in enzyme dilution buffer containing 1 mM n-dodecyl β-D-maltoside (Sigma-Aldrich), as a detergent. A control sample was suspended in 0.2 mg/mL in enzyme dilution buffer alone. Samples were incubated at 4° C. for 10 minutes, and then 20 μL of each sample was added to cuvettes containing 950 μL of assay buffer (10 mM Tris-HCl, 120 mM KCl, pH 7.0) and 80 μL of enzyme dilution buffer. The cuvettes were placed into a spectrophotometer. The assay was started by adding 50 μL of ferrocytochrome c substrate (Sigma-Aldrich) (0.22 mM, reduced by dithiothreitol (DTT) at a final concentration of 0.5 mM) to cuvettes and mixing by pipetting. Absorbance (A⁵⁵⁰) was read at 5, 15, 25, 35, and 45 sec after the addition of ferrocytochrome c substrate. A graph of A⁵⁵⁰ as a function of time was plotted to calculate the maximum linear rate (A/s) for both sample and control. The degree of mitochondrial integrity was calculated by the Equation 1 below.

$$\% MOM = \frac{Δ A / s ( with detergent ) - Δ A / s ( without detergent )}{Δ A / s ( with detergent )} \times 100 \% \qquad ( Equation 1 )$$

$$where Δ A / s = A / s ( sample ) - A / s ( blank ) \qquad ( Equation 2 )$$

### Transmission Electron Microscopy (TEM)

Samples of mitochondria, before and after filtration, were fixed with glutaraldehyde 2.5%, stained with uranyl acetate (Sigma-Aldrich), and embedded in plastic, sectioned (Ultramicrotome™ Leica EM UC7, Leica Biosystems, Buffalo Grove, Ill.) and examined by TEM on a Hitachi H-7650 transmission electron microscope (EM) (Hitachi, Tokyo). The numbers of intact mitochondria were quantitated per field using ImageJ.exe and expressed as means±standard deviation (SD) per field at identical magnifications.

### Respiration Assay

The respiration mitochondria as determined by oxygen consumption was assayed using Seahorse analyzer (XF24, Seahorse Bioscience, North Billerica, Mass.) as described in the Seahorse isolated mitochondria respiration assay protocol. Mitochondria, 8 μg measured by BCA protein assay (Pierce, Rockford, Ill.) were placed in each well, and exposed sequentially to adenosine diphosphate (ADP), oligomycin, carbonyl cyanide-4-(trifluoromethoxy)phenylhydrazone (FCCP), and antimycin. Oxygen consumption was expressed in units of pmole/min normalized to the amount of mitochondrial DNA present as determined by qPCR and expressed as means±SD.

### Results

Table 1 shows laser light scattering data on particle size and charge at various stages of mitochondrial preparation. The mean particle diameter of after the low speed was 295 nm reflecting elimination of large impurities. After the first and second high speed spins, the mean particle diameters increased to 409 nm and 566 nm, respectively, reflecting elimination of small debris. Electron microscopy (EM) images of mitochondria preparations after high-speed spins still showed considerable evidence of membrane fragments. For this reason, the effects of filtration were studied. Table 1 shows that filtration through 1.2μ and 0.8μ membranes progressively decreased mean particle diameter to 536 nm and 520 nm, respectively.

| | Particle | Mitochondrial DNA | Mitochondrial DNA |
|---|---|---|---|
| Purification | Size | (fold-change/ | (mitochondrial copy |
| Step | (nm) | LDHA) | number/LDHA) |
| 1000 X g spin | 300 ± 10 | 1.0 | 1200 ± 270 |
| (Supernatant) | | | |
| First 7000 X g | 410 ± 10 | 1.3 | 1590 ± 390 |
| spin (Pellet) | | | |
| Second 7000 X g | 570 ± 20 | 1.7 | 1990 ± 420 |
| spin (Pellet) | | | |
| Filtrate | 540 ± 20 | 4.2 | 5050 ± 1400 |
| (1.2μ) | | | |
| Filtrate | 520 ± 10 | 6.7 | 8010 ± 1590 |
| (0.8μ) | | | |

### Mitochondrial DNA

To quantitate the effects of the purification steps, mitochondrial DNA was measured by quantitative PCR analysis by amplifying a specific region of 116 bp relative to LDHA, which is a marker of genomic DNA contamination, and those sequences are absent in mitochondrial DNA. As shown in Table 1, mitochondrial DNA was significantly purer after each step of filtration. The high and low speed spins resulted in a 1.7-fold increase in mitochondrial DNA over genomic contamination. The additional filtration steps increased the ratio 6.7-fold. To provide a better comparison in terms of amount of mitochondrial DNA at each step, the mitochondrial to LDH copy number ratio increased from 1200 to 8000 after that last filtration step. The data indicate that the sequential filtration steps significantly improve the mitochondrial purity compared to the standard method of varying speed spins alone.

### Integrity of Mitochondria Outer Membrane (MOM)

In order to assess if the mitochondria were intact after passing through the filters, cytochrome c (cty c) activity assay was used to test for the integrity of the mitochondria outer membrane. The values obtained showed that 95%±3.1% of the isolated mitochondria remained intact after the final filtration step.

### Mitochondrial Function

While the cytochrome c activity assay showed that the isolated mitochondria remained intact after filtration, other damage could have occurred during the purification steps, especially after the pressure applied during the sequential filtration steps. To determine whether the filtration resulted in changes to mitochondrial function, mitochondrial respiration function was measured. FIG. 1 shows that the respiration the mitochondria after the standard high-speed spin preparation was indistinguishable from that of mitochondria purified by sequential filtration. In particular, in mitochondria before filtration, activation by ADP increased oxygen consumption rate (OCR) of from 170 to 530 pmol/min. Similarly, for mitochondria after spins and dual sequential filtration, activation by ADP increased OCR of from 180 to 540 pmol/min. Oligomycin decreased OCR for mitochondria from high speed spins alone and spins plus filtration by about 30%. Release by FCCP, and final inactivation by antimycin, all characteristic of mitochondrial oxygen consumption were not significantly different. The data suggest that the mitochondrial samples retained their energy generating function, and the filtrations did not significantly alter mitochondrial respiratory function.

### Electron Microscopy

Electron micrograph analysis was performed to determine whether there were visible differences in the mitochondrial preparations purified by standard high-speed spins alone (before filtration), and those after spins and sequential filtration. FIG. 2A shows that at 5000×there was a considerable amount of large and small membranous debris were found in filtered mitochondria compared mitochondria before filtration. In contrast, there were much less membranous debris after filtrations (FIG. 2B). Furthermore, as seen in FIG. 2C, the mean number of mitochondria identified per field after spins alone was 17, while the mean number of mitochondria per field was measured as 50. Thus, there is a significant difference between preparations only centrifuged compared to sequential centrifugation and filtration.

The low and high-speed spin steps based on the method previously described by Frezza et al. Filtration has been described previously to eliminate relatively large particulate matter from mitochondrial preparations. For example, Preble et al. (“Rapid isolation and purification of mitochondria for transplantation by tissue dissociation and differential filtration.” J Vis Exp 2014:e51682) used filtration through sequential 40μ and 10μ nylon filters, followed by a high-speed spin for final purification of mitochondria from fresh muscle tissue. Since the mean diameter of mitochondria has been reported to be between 500 nm and 1000 nm, these membrane filters allow mitochondria to pass though while removing large debris such as incompletely lysed cells, and large organelle aggregates. In this experiment, much more restrictive 1.2μ and 0.8μ filters were selected to increase the purity of preparations, thereby maximizing the elimination of debris larger than mitochondria. In fact, some large mitochondria may have been lost during the filtration process. However, these filters were the best option because only the purest, smallest, and most uniform populations of mitochondria were of most interest. However, if uniformly small mitochondria are not needed, a single filtration through 1.2μ filter would improve yield. Nevertheless, this is the first description of a mitochondrial isolation method using simple rapid spin steps and a sequential sub-1.2μ filtration to provide isolated uniform highly purified, and functional mitochondria from liver tissue.

### Example 2: Targeted Delivery of Mitochondria to the Liver in Rats

### Methods

### Animals

Animal care and use were in accordance with NIH animal use guidelines. Female CD-1 mice (3-weeks old) and female Sprague-Dawley rats (6- to 7-weeks old) were purchased from the Charles River Laboratories and maintained in 12-h light-dark cycles with food and water available ad libitum. At day 23 after birth, mice were killed, and mitochondria were isolated from the liver as described below.

### Isolation of Mitochondria

To distinguish donor from recipient mitochondria, CD-1 mice were used as mitochondrial donors and Sprague-Dawley rats as recipients. Specific primers were developed for mouse and rat mitochondrial DNA to permit polymerase chain reaction (PCR) capable of completely distinguishing between the two. Donor liver mitochondria were prepared in isolation buffer (IB) (0.01-M tris-4-morpholinepropanesulfonic acid, 0.01-M ethylene-bis (oxyethylenenitrilo)tetraacetic acid, 0.2-M sucrose, pH 7.4) using homogenization, centrifugation at 1000×g, twice at 7000×g, and filtered through sterile Acrodisc™ 1.2 and 0.8μ syringe filters (Pall Corp, Ann Arbor, Mich., USA) immediately before use. Preparations were used immediately or stored on ice and used within 3 h. The purity was determined by quantitative polymerase chain reaction (qPCR) of mouse mitochondria DNA and transmission electron microscopy. Mitochondrial integrity was assessed by cytochrome c assay (Sigma-Aldrich, St. Louis, Mo., USA).

### Preparation of Asialoglycoprotein Conjugates

Orosomucoid was prepared from human serum (American Red Cross) and desialylated to form asialoorosomucoid (AsOR).^{9,10} To prepare a hepatocyte-targetable carrier protein, AsOR was chemically coupled to poly-L-lysine (PL) (Sigma-Aldrich, St. Louis, Mo., USA) in a 1:10 molar ratio to form AsOR-PL conjugate using a cross-linker, 1-ethyl-3-(3-dimethylaminopropyl) carbodiimide hydrochloride. The product was purified using an Amicon™ centrifugal filter (10 000 molecular weight exclusion) (Sigma-Aldrich, St. Louis, Mo., USA).

### Formation of Targetable Mitochondrial Complexes

Purified mitochondria in IB were mixed in a 1:0.02-1:0.025 ratio based on A²⁸⁰ of the samples in a volume of 500 μL. The size and charge of all mitochondrial samples were determined using a laser light scattering particle size analyzer (Model #90 Plus; Brookhaven Instruments, Holtsville, N.Y., USA). Mitochondrial complexes for injection typically had a mean diameter of 500 nm and a charge of −2.49, while control mitochondria alone had a mean diameter of 444 nm and a charge of −18.8.

### Assay for Mitochondrial Integrity

The integrity of mitochondrial outer membrane (MOM) was determined by a cytochrome c oxidase assay kit (Sigma-Aldrich, St. Louis, Mo., USA) (Farah N. et al. “An improved method for preparation of uniform and functional mitochondria from fresh liver.” J. Clin Transl Hepatol 2019; 7:46-50). Mitochondria or complexed mitochondria were suspended in 0.2 mg/mL in enzyme dilution buffer containing 1 mM n-dodecyl β-D-maltoside (Sigma-Aldrich, St. Louis, Mo., USA). A control sample was suspended in 0.2 mg/mL in enzyme dilution buffer alone. After incubation, samples were added to cuvettes containing assay buffer (10-mM Tris-HCl, 120-mM KCl, pH 7.0) and enzyme dilution buffer.

Ferrocytochrome c substrate (Sigma-Aldrich, St. Louis, Mo., USA) (0.22 mM, reduced by dithiothreitol at a final concentration of 0.5 mM) was added and absorbance (A⁵⁵⁰) was read at 5 s, 15 s, 25 s, 35 s, and 45 s after the addition of ferrocytochrome c substrate. A graph of A⁵⁵⁰ as a function of time was plotted to calculate the maximum linear rates (A/s). The degree of mitochondrial integrity was calculated (Farah N. et al.).

### Preparation of Listeriolysin O (LLO) Conjugates

Listeriolysin O (LLO) was prepared from an Escherichia coli strain containing a plasmid encoding LLO with a his tag (Gedde M. et al. “Role of listeriolysin O in cell-to-cell spread of Listeria monocytogenes.” Infect Immun 2000; 68:999-1003). An endosomal-escape reagent capable of being co-internalized with complexed mitochondria was prepared by chemically linking LLO to AsOR by disulfide bonds using succinimidyl 3-(2-pyridyldithio)propionate (SPDP) (ThermoFisher, MA, USA). AsOR was linked to SPDP with weight ratio of 2:1 in 0.05-M sodium acetate (pH 5.5) for 1 h at room temperature. AsOR-SPDP was desalted with a PD10 column (GE Healthcare, Little Chalfont, UK) and incubated with DTT-treated his-tagged LLO in a molar ratio of 2:1, in 0.05-M sodium acetate buffer (pH 6.5) overnight at 4° C. The AsOR-LLO conjugate was purified by a diethylaminoethyl sephacel column (Thermo Fisher Scientific, Waltham, Mass., USA) equilibrated with 0.05-M sodium acetate buffer, pH 6.0. The column was washed with 4× the void volume of 0.05-M sodium acetate, pH 6.0, and eluted using 0.05-M sodium acetate, pH 4.3. The eluate was concentrated and equilibrated with 50-mM sodium phosphate buffer, pH 8.0, 300-mM sodium chloride, and 10-mM imidazole (Sigma-Aldrich, St. Louis, Mo., USA) and applied onto a 1 mL nickel-agarose column (Qiagen, Hilden, Germany). Unreacted AsOR was removed with 8-mL lysis buffer followed by 8 mL of 50-mM sodium phosphate buffer, pH 8.0, 300-mM sodium chloride, and 20-mM imidazole. Purified AsOR-LLO conjugate was eluted with 4 mL of elution buffer (50-mM sodium phosphate buffer, pH 8.0, 300-mM sodium chloride, and 250-mM imidazole). All AsOR-LLO samples used in subsequent experiments showed acid-dependent hemolytic properties (Koster S. et al. “Crystal structure of listeriolysin O reveals molecular details of oligomerization and pore formation.” Nat Commun 2014; 5:1-4) under reducing conditions known to be present in endosomes.

### Respiration Assay

Mitochondrial respiration was determined by oxygen consumption assay using the Seahorse Analyzer XF24 (Seahorse Bioscience, North Billerica, Mass., USA).⁸ Mitochondria, 8 measured by BCA protein assay (Pierce, Rockford, Ill., USA) were exposed sequentially to adenosine diphosphate (ADP), oligomycin, carbonyl cyanide-4 (trifluoromethoxy)phenyl hydrazone (FCCP), and antimycin. Oxygen consumption was expressed in units of pmol/min normalized to the amount of mitochondrial DNA present as determined by qPCR and expressed as means±SD.

### Animal Infusions

Sprague-Dawley rats weighing 250 g were anesthetized with ketamine/xylazine, and 23 G catheters (Becton-Dickinson, NJ, USA) were placed in tail veins. A peristaltic pump was employed to deliver agents in 0.5 mL of IB at a steady rate of 160 μL/min. Rats received equal numbers of mitochondria in the form of mitochondria alone, complexed mitochondria alone, or complexed mitochondria plus AsOR-LLO, the latter in a (A²⁸⁰) ratio of 500:1. Each group was studied at least in triplicate, and most experiments were repeated more than once. After 1 h, in some experiments 2 h and 24 h, animals were killed, and organs were harvested, weighed, and sectioned. Portions of organ samples of known weight were homogenized for DNA extraction or fixed for in situ PCR, hybridization, and immunohistochemistry.

### Mitochondrial DNA Analyses

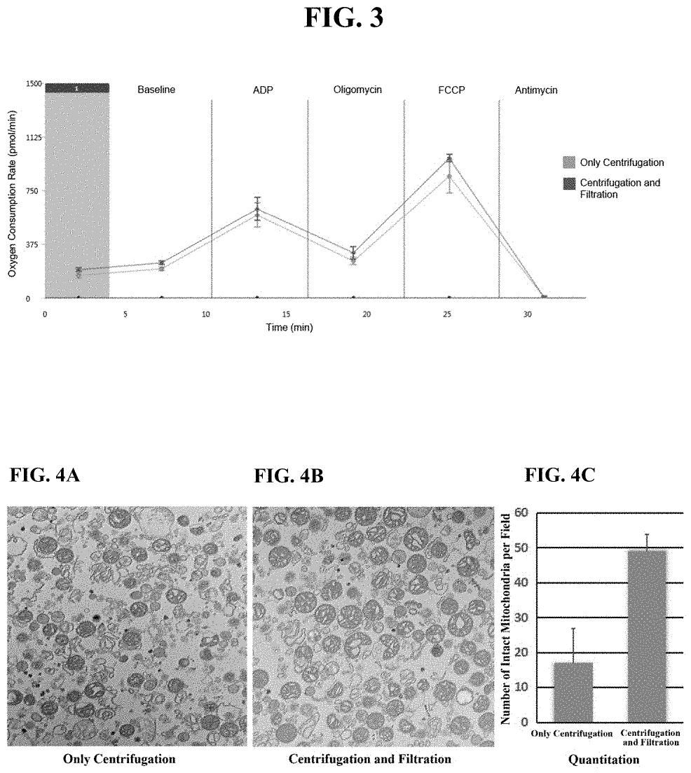

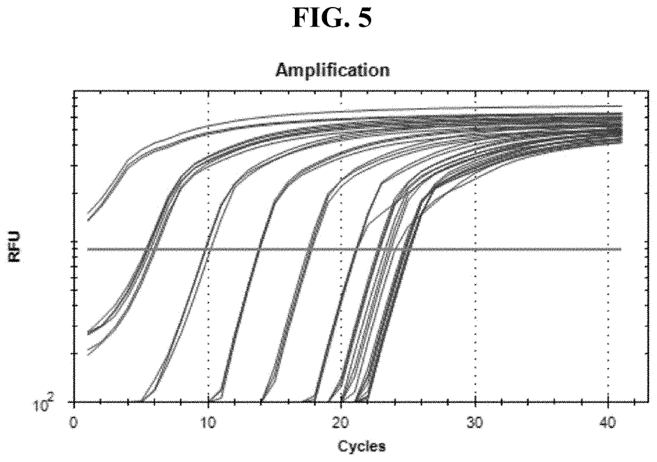

Quantitative PCR was used to analyze mitochondrial DNA extracted from isolated mitochondria (DNeasy™ Kit, Qiagen, the Netherlands). Mitochondrial DNA (mtDNA), 100 ng, was used as a template for qPCR with primers designed to amplify a specific region of mouse mitochondria 116 bp in length (primers: 5′-TCGCCTACTCCTCAGTTAGCCACA-3′(SEQ ID NO: 1), 5′-TGATGATGTGAGGCCATGTGCGA-3′(SEQ ID NO: 6), Integrated DNA Technology, Skokie, Ill.). ΔΔCt values were calculated to compare mtDNA with lactate dehydrogenase A (LDHA) signals, the latter as a measure of cell nuclear DNA contamination (primers: 5′-TAATGAAGGACTTGGCA GATGAACT-3′(SEQ ID NO: 7), 5′-ACGGCTTTCTCCCTCTTGCT-3′(SEQ ID NO: 8), Integrated DNA Technology). Copy number was determined by preparation of a specific sequence of mouse mitochondrial DNA (5′-TCGCCTACTCCTCAGTTAGCCACATAGCACTTGTTATTGCATCAATCATAATCCAAAC TCCATGAAGCTTCATAGGAGCAACAATACTAATAATCGCACATGGCCTCACATCATC A-3′(SEQ ID NO: 9)), which was amplified to generate products, known amounts of which were used to produce a standard curve for quantification. (FIGS. 3-5B).

### Transmission Electron Microscopy

Samples of mitochondria before and after complexation were fixed with glutaraldehyde 2.5%, stained with uranyl acetate (Sigma-Aldrich, St. Louis, Mo., USA), embedded in plastic, sectioned (Ultramicrotome™ Leica EM UC7, Leica Biosystems, Buffalo Grove, Ill., USA), and examined by transmission electron microscopy on a Hitachi H-7650 transmission electron microscope (Hitachi, Tokyo, Japan). The numbers of intact mitochondria were quantified per field using ImageJ.exe and expressed as means±SD per field at the same magnifications.

### In Situ Polymerase Chain Reaction and In Situ Hybridization

Donor mouse mitochondrial DNA was amplified in situ and hybridized in situ to labeled probes specific to the amplified products to determine the location and cell types containing the transplanted mitochondrial DNA. Liver tissues were fixed in 10% formalin for 12 h and then embedded in paraffin. Sections 0μ thick were placed on poly-1-lysine-coated glass slides. Prior to PCR, sections were digested by 2 mg/mL pepsin for 30 min. Then, in situ PCR was carried out using primers designed to amplify a specific region of mouse mitochondrial DNA (primers: 5′-TCGCCTACTCCTCAGTTAGCCACA-3′ (SEQ ID NO: 1), 5′-TGATGATGTGAGGCCATGTGCGA-3′ (SEQ ID NO: 2), Integrated DNA Technology, Skokie, Ill., USA). A digoxigenin (DIG)-labeled mitochondrial DNA probe specific for mouse mitochondrial DNA was created by adding DIG-dUTP (Biotium, Fremont, Calif., USA) to the PCR reaction buffer with mitochondrial DNA as template (primers: 5′-TCGCCTACTCCTCAGTTAGCCACA-3′ (SEQ ID NO: 1), 5′-TGATGATGTGAGGCCATGTGCGA-3′ (SEQ ID NO: 2), Integrated DNA Technology, Skokie, Ill., USA). After in situ PCR, the DIG-labeled mitochondria DNA probe was added to sections and incubated at 95° C. for 10 min followed by 37° C. overnight. Sections were incubated overnight with rabbit anti-DIG antibody (1:100) (Invitrogen, CA, USA) followed by horseradish peroxidase-conjugated goat anti-rabbit IgG (1:2000) (ThermoFisher, MA, USA). A chromogen, 3,3′-diaminobenzidine (DAB) (Invitrogen), was used to develop signals. Nuclei were visualized by hematoxylin staining. Samples were imaged using an Olympus BX60 microscope (Olympus, Tokyo, Japan) at 200× and 400× magnifications.

### Statistical Analysis

Values were expressed as means±SD. One-way analysis of variance followed by Tukey's multiple comparison test was performed for multiple sample comparisons using SPSS statistics software version 19.0. Differences were considered statistically significant when P<0.05.

### Results

### Integrity of Mitochondria in Complexes

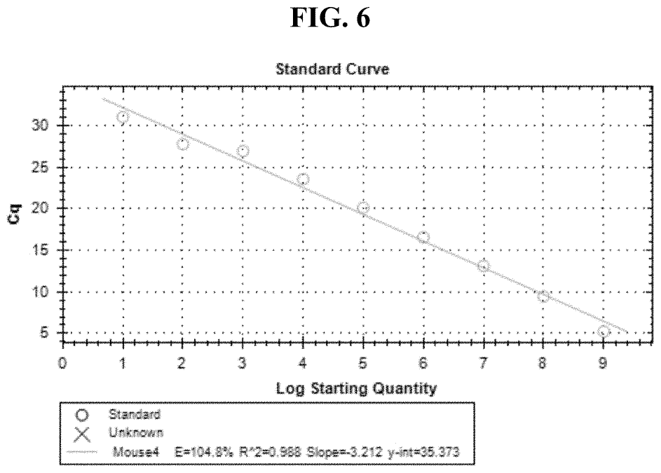

Mitochondria were significantly purified by high-speed spins followed by filtration (see Example 1). Transmission electron microscopy of mitochondrial preparations before complexation (FIG. 6A) mostly intact mitochondria within a narrow size range. Addition of AsOR-PL conjugate to form mitochondrial complexes (FIG. 6B) showed that mitochondria retained normal shape and uniformity of size. There were no visible changes in the surface membranes or obvious increase membranous debris in complexed preparations, although the density of mitochondria in the latter appeared to be slightly greater than the non-complexed mitochondria. Particle size analysis revealed that structures characterized by multiples of mitochondrial diameters were not present arguing against the presence of mitochondrial aggregation.

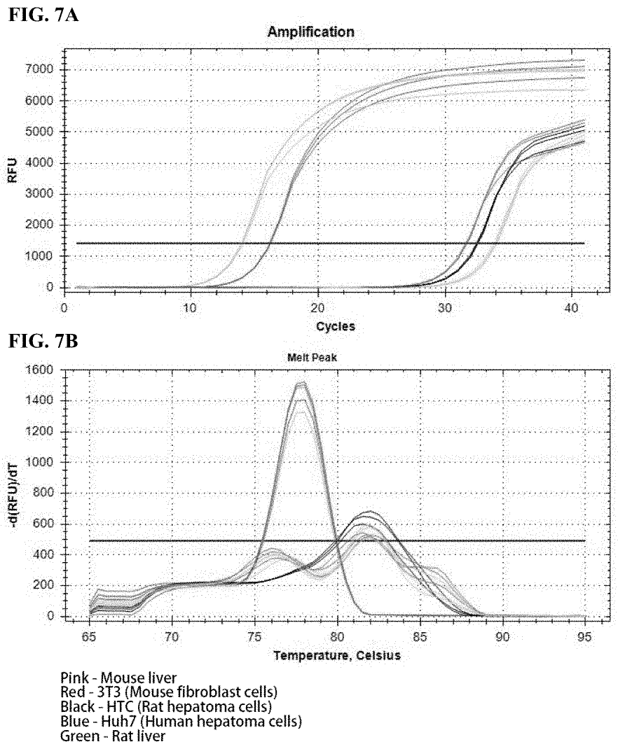

The cytochrome c oxidase assay revealed no significant differences in intactness of MOM before and after complexation (96.5% and 92.1%, respectively) (FIG. 7A) when samples were incubated in IB, the buffer selected specifically to preserve mitochondrial integrity and function. Some cell culture media like Dulbecco's™ modified eagle medium (DMEM) are known to damage mitochondria (Corcelli A. et al. “Mitochondria isolated in nearly isotonic KCl buffer: focus on cardiolipin and organelle morphology.” Biochim Biophys Acta 2010; 1798:681-87; Torralba D. et al. “Mitochondria know no boundaries: mechanisms and functions of intercellular mitochondrial transfer.” Front Cell Dev Biol 2016; 4:107). Cytochrome c assay data confirmed this as the integrity of MOM of mitochondria alone in DMEM was only 74.5%, more than 20% lower than in IB. This is consistent with previous reports that mitochondria are more stable in buffers containing nearly iso-osmotic sucrose (e.g., IB) than those that do not (Corcelli A. et al.; Hogeboom G H, Schneider W C, Pallade G E. Cytochemical studies of mammalian tissues; isolation of intact mitochondria from rat liver; some biochemical properties of mitochondria and submicroscopic particulate material. J Biol Chem 1948; 172:619-35). However, the MOM integrity of AsOR-PL complexed mitochondria in DMEM under the same conditions was 90.4% (FIG. 7A), similar to results obtained in IB.

FIG. 7B shows that mitochondria alone and AsOR-PL-mitochondria complexes had nearly identical baseline oxygen consumption rates of 42 and 44 pmol/min, respectively. Upon addition of ADP, the oxygen consumption rate of mitochondria alone rose to 48.5, while that of complexed mitochondria increased to 57.4 pmol/min. This difference was not significant (P>0.05). Addition of various inhibitors resulted in a pattern of changes that was virtually the same qualitatively for both and without significant quantitative differences.

### Evidence of Targeted Transplantation of Mitochondria in Rats

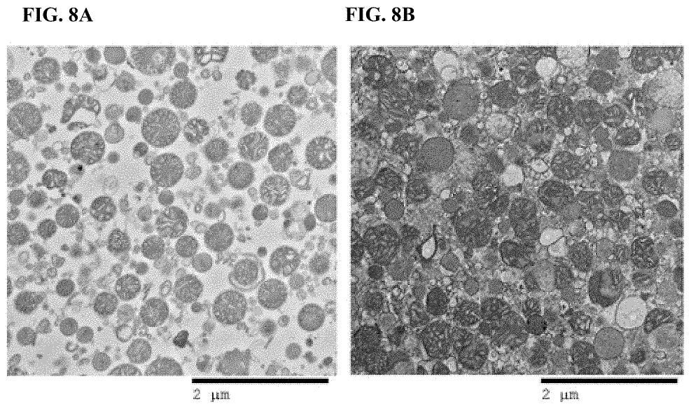

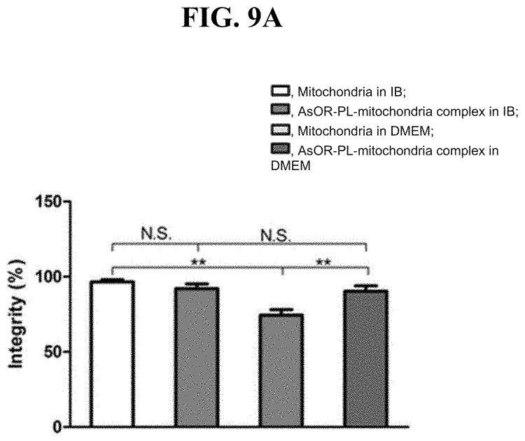

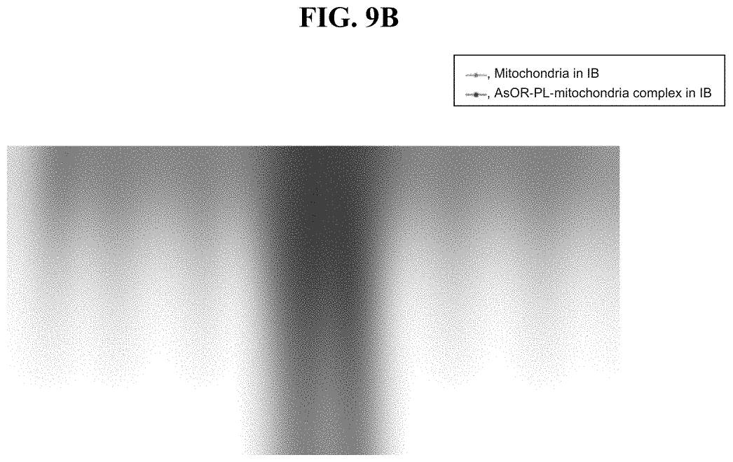

Mouse mitochondria equivalent to 1×10⁹ copies of mouse mitochondria DNA were injected intravenously into each rat. FIG. 8 shows that in the mitochondria alone group, 2.7% of injected mitochondrial DNA was present in livers. However, injection of complexed mitochondria more than tripled that fraction to 9.2%. Furthermore, complexed mitochondria co-injected with AsOR-LLO further increased the fraction to 27.1%, a threefold and significant increase over complexed mitochondria alone (P<0.01) in the liver. The fraction of mitochondria delivered did not exceed 2% to spleen or 1% to the lungs for any mitochondrial preparation. However, the results could have been to differences in the number of cells per organ and not the numbers of mitochondria per cell. To determine whether there was an actual increase in donor mitochondrial DNA per cell, the mitochondria DNA fold-changes were measured relative to LDHA (to normalize to the number of recipient cells per sample). FIG. 9 shows that in the liver, the fold-change was 3.9-fold for mitochondria alone, 15.8-fold for the complex group, and 40.1-fold in complex+AsOR-LLO group, the latter two were significantly different from mitochondria alone and each other (P<0.01). In contrast, the fold-change in spleen was only about 4.0-fold for complexed mitochondria. Co-injection with AsOR-LLO did not result in any significant change in the DNA level compared with complex alone. In the lungs, complexed mitochondria produced only a 6.0-fold change. Co-injection of AsOR-LLO did not change this value, although both values were higher than that for mitochondria alone which was about 3.0-fold (FIG. 9). These non-uniform organ distribution data suggest that the observed uptake in the liver was not due to a generalized non-specific uptake process and that the effect of co-injection of AsOR-LLO was restricted to the liver. FIG. 9 shows the results of quantification of donor mouse DNA in recipient rat livers at 2 and 24 h after injection of donor mouse mitochondria in the form of mitochondria alone, mitochondria complex, and mitochondria complex plus AsOR-LLO. The results showed that the differences between complexes alone and complexes plus AsOR-LLO tended to decrease with time. Without being bound to theory, one of the possible reasons to explain this may be the saturation of the receptor or the competition for receptor binding between the two AsOR-containing entities. Nevertheless, the data demonstrate that large numbers of injected mitochondria in the liver remained detectable in the liver at least 24 h after injection.

### Cellular and Intracellular Localization of Transplanted Mouse Mitochondria

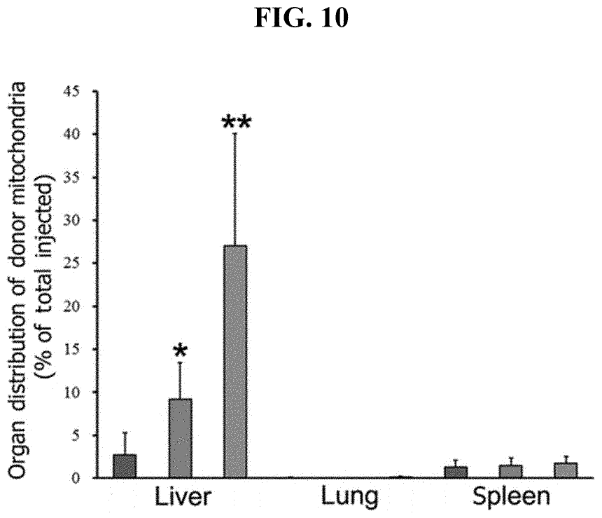

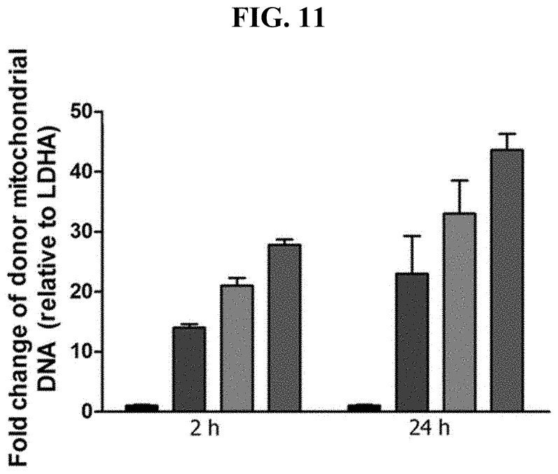

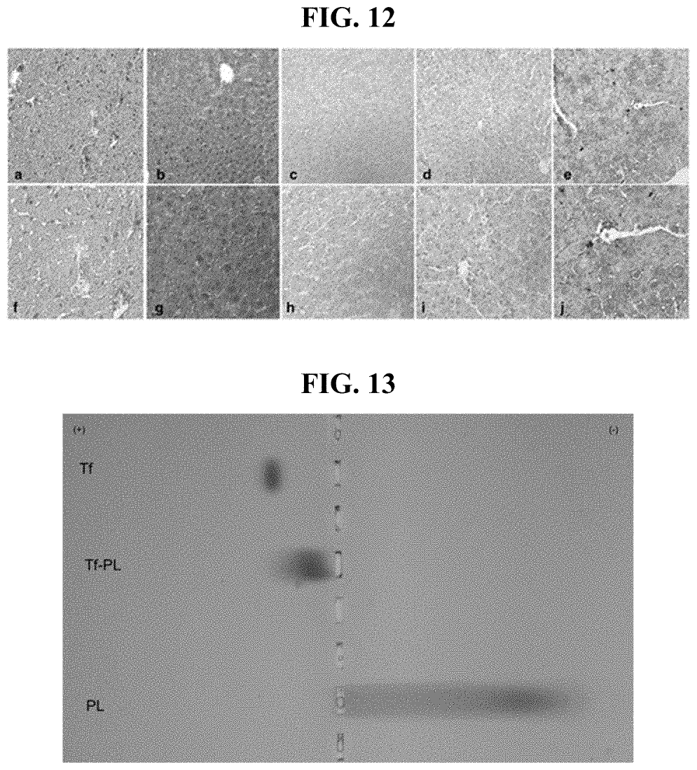

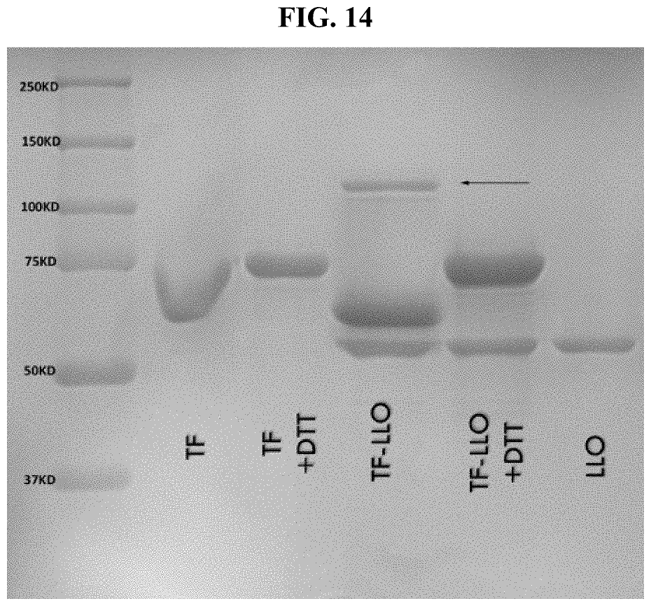

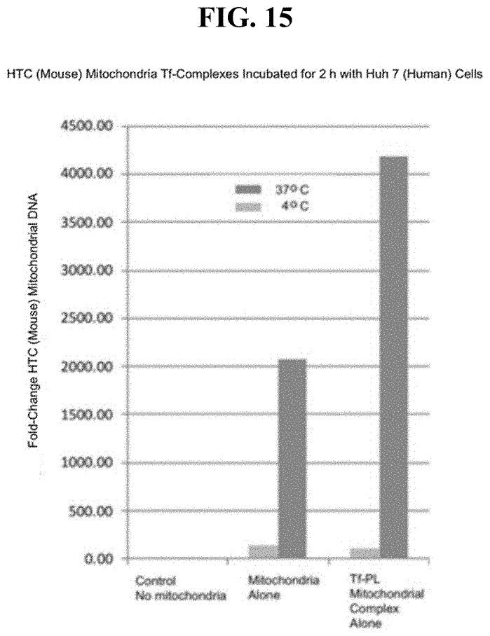

Mouse mitochondrial DNA was amplified by in situ PCR and in situ hybridization and detected by a digoxigenin (DIG)-labeled DNA probe specific for mouse mitochondrial DNA. The signals were developed by anti-digoxygenin antibody-horseradish peroxidase followed by immunohistochemical staining. As showed in FIG. 10, control (non-injected) rat liver (FIG. 10A and FIG. 10F) showed no staining. Livers from rats injected with mouse mitochondria alone (FIG. 10C and FIG. 1011), also showed no obvious staining. Injection of complexed mitochondria alone (FIG. 10D and FIG. 10I), resulted in a low level of staining in recipient rat liver. In contrast, injection of complexed mitochondria plus AsOR-LLO had strong staining (FIG. 10E and FIG. 10J). In the latter group, the staining appeared to be more intense in periportal compared with centrilobular areas but was not uniformly distributed as seen in normal mouse liver (FIG. 10B). FIG. 10J at 400× magnification showed that staining was clearly present in hepatocytes and not in non-parenchymal cells. Furthermore, the perinuclear location is similar to that noted for normal native mitochondria. No animals showed any signs of respiratory distress or failure before sacrifice, and all animals survived until sacrifice. Histological examination of all sampled organs showed no gross evidence of toxicity.

In this experiment, the initial contact of mitochondria with hepatocytes was expected to be with periportal cells. It is interesting to note that the most intense staining of donor mitochondria was detected in periportal areas. However, the regional distribution was not uniform, which suggests a non-uniform delivery of mitochondria to the hepatic vessels.

Several methods have been described to achieve mitochondrial transfer between cells. For example, cell fusion (Torralba D. et al.), microvesicles (Paliwal S. et al. “Regenerative abilities of mesenchymal stem cells through mitochondrial transfer.” J Biomed Sci 2018; 25:31), gap junction (Li H. et al. “Mitochondrial transfer from bone marrow mesenchymal stem cells to motor neurons in spinal cord injury rats via gap junction.” Theranostics 2019; 9:2017-35), and tunneling nanotube formation (Lu J. et al. “Tunneling nanotubes promote intercellular mitochondria transfer followed by increased invasiveness in bladder cancer cells.” Oncotarget 2017; 8:15539-52) have been documented. All these processes of mitochondrial transfer are non-specific. As a result, most of the mitochondria transplantation experiments carried out previously have been non-specific in terms of recipient cell type. There are several genetic liver diseases known to be caused by defects in mitochondria. These include mitochondrial depletion syndrome and ornithine transcarbamylase deficiency. Furthermore, mitochondrial damage and dysfunction are also common mechanisms for the development of acquired liver disease (Ramachandran A. et al. “Mitochondrial dysfunction as a mechanism of drug-induced hepatotoxicity: current understanding and future perspectives.” J Clin Transl Res 2018; 4:75). Although the objective of the current studies was to determine short-term effects of mitochondrial transplantation, it is possible that long-term benefits may occur because it is possible that donor mitochondria could replicate in recipient cells and be transferred from parent to daughter cells during cell division. This scenario is especially likely to occur in a regenerative or recovery phase of liver injury when the rates of hepatocyte cell division are high (Li H. et al. “Mitochondrial transfer from bone marrow mesenchymal stem cells to motor neurons in spinal cord injury rats via gap junction.” Theranostics 2019; 9:2017-35; Roushandeh A. et al. “Mitochondrial transplantation as a potential and novel master key for treatment of various incurable diseases.” Cytotechnology 2019; 71:647-63; Hayakawa K. et al. “Protective effects of endothelial progenitor cell-derived extracellular mitochondria in brain endothelium.” Stem Cells 2018; 36:1404-10). Thus, the ability of mitochondria to replicate to meet cell needs makes it theoretically possible that a single administration of only few transplanted mitochondria per cell could be sufficient to restore the normal complement of mitochondria. Recently, mitochondrial transplantation has been suggested as potential treatment for a variety of other diseases (Shin B, Cowan D B, Emani S M, Del Nido P J, McCully J D. Mitochondrial transplantation in myocardial ischemia and reperfusion injury. Adv Exp Med Biol 2017; 982:595-19; Masuzawa A, Black K M, Pacak C A et al. Transplantation of autologously derived mitochondria protects the heart from ischemia-reperfusion injury. Am J Physiol Heart Circ Physiol 2013; 304:H966-82; Chang J C, Chang H S, Wu Y C et al. Mitochondrial transplantation regulates antitumour activity, chemoresistance and mitochondrial dynamics in breast cancer. J Exp Clin Cancer Res: CR 2019; 38:30).

1. A mitochondrial-protein complex comprising:
isolated mitochondria; and
asialoorosomucoid-poly-L-lysine (AsOR-PL) conjugate or transferrin-poly-L-lysine (Tf-PL) conjugate.

2. The mitochondrial-protein complex of claim 1, having a mean particle diameter of about 500 nm.

3. The mitochondrial-protein complex of claim 1, wherein the isolated mitochondria are extracted from mammalian cells.

4. The mitochondrial-protein complex of claim 3, wherein the mammalian cells are liver cells.

5. The mitochondrial-protein complex of claim 3, wherein the mammalian cells are selected from human cells, porcine cells, canine cells, rodent cells, and feline cells.

6. The mitochondrial-protein complex of claim 3, wherein the mammalian cells are rodent cells.

7. (canceled)

8. A pharmaceutical composition comprising the mitochondrial-protein complex of claim 1.

9. The pharmaceutical composition of claim 8, wherein the pharmaceutical composition comprises one or more pharmaceutically acceptable excipients.

10. A method of transplanting mitochondria into subject, wherein the method comprises administering the mitochondrial-protein complex of claim 1, to a subject in need thereof.

11.-12. (canceled)

13. A method of treating a mitochondrial dysfunctional disorder, wherein the method comprises administering a mitochondrial-protein complex of claim 1 to a subject in need thereof.

14. The method of treating a mitochondrial dysfunctional disorder of claim 13, wherein the mitochondrial dysfunction disorder is a neuropsychiatric disorder.

15. (canceled)

16. The method of treating a mitochondrial dysfunctional disorder of claim 13, wherein the mitochondrial dysfunction disorder is a neurodegenerative disorder or diabetes.

17.-20. (canceled)

21. The method of treating a mitochondrial dysfunctional disorder of claim 13, wherein the mitochondrial dysfunctional disorder is mitochondrial toxicity associated with therapeutic agents.

22.-25. (canceled)

26. The method of treating a mitochondrial dysfunctional disorder of claim 13, wherein the mitochondrial dysfunctional disorder is a migraine.

27. The method of treating a mitochondrial dysfunctional disorder of claim 13, wherein the mitochondrial dysfunctional disorder is an ocular disorder associated with mitochondrial dysfunction.

28. (canceled)

29. The method of treating a mitochondrial dysfunctional disorder of claim 13, wherein the mitochondrial dysfunctional disorder is an ischemia related condition.

30. (canceled)

31. A method of
(a) increasing muscle performance, wherein the method comprises administering a mitochondrial-protein complex comprising:
isolated mitochondria; and
transferrin-poly-L-lysine (Tf-PL) conjugate,
to a subject in need thereof; or
(b) treating a liver disease, wherein the method comprises administering a mitochondrial-protein complex and AsOR-listeriolysin (LLO) to a subject in need thereof, wherein the mitochondrial-protein complex comprises:
isolated mitochondria; and
asialoorosomucoid-poly-L-lysine (AsOR-PL) conjugate.

32.-36. (canceled)

37. The composition of claim 1, further comprising an endosomolytic escape protein.

38.-39. (canceled)

40. The composition of claim 37, wherein the composition further comprises listeriolysin or another targetable endosomolytic agent.

41.-47. (canceled)

48. A method of preparing a mitochondrial-protein complex comprising mixing
isolated mitochondria; and
asialoorosomucoid-poly-L-lysine (AsOR-PL) conjugate or transferrin-poly-L-lysine (Tf-PL) conjugate.

49.-61. (canceled)
